(19) United States
(12) Patent Application Publication
(10) Pub. No.: US 2012/0213707 A1
(43) Pub. Date: Aug. 23, 2012

(54) Riboflavin Based Aerosol and Use as Placebo in Trials

(75) Inventor: David C. Griffith, San Marcos, CA (US)

(73) Assignee: MPEX PHARMACEUTICALS, INC., San Diego, CA (US)

(21) Appl. No.: 13/398,507

(22) Filed: Feb. 16, 2012

Related U.S. Application Data
(63) Continuation of application No. PCT/US2010/002306, filed on Aug. 19, 2010.
(60) Provisional application No. 61/235,319, filed on Aug. 19, 2009.
(60) Provisional application No. 61/249,228, filed on Oct. 6, 2009.

(51) Int. Cl. A61K 31/675; A61P 31/04; A61P 37/08; A61P 35/00; A61P 31/10; A61P 29/00; A61P 37/02; A61P 37/04; A61P 11/14; A61P 11/08; A61P 11/04; A61K 9/00; C12Q 1/06; C12Q 1/56; A61K 49/00

(52) U.S. Cl. 424/9.2; 514/81; 424/400; 435/39; 435/13

(57) ABSTRACT

A method for evaluating an aerosolized test compound includes administering a test compound to a first population of individuals, via inhalation of an aerosol; administering a placebo comprising riboflavin 5′-phosphate to a second population of individuals, via inhalation of an aerosol; and comparing a biological marker for the individuals in the two populations. An aerosol comprising riboflavin 5′-phosphate (also known as flavin mononucleotide) may be used either as a placebo in clinical trials, or therapeutically.

### RELATED APPLICATIONS

This application is a continuation of International Patent Application No. PCT/US2010/002306 filed Aug. 19, 2010 which claims the benefit of priority under 35 U.S.C. §119 to U.S. Provisional Application No. 61/235,319, filed Aug. 19, 2009, and U.S. Provisional Application No. 61/249,228, filed Oct. 6, 2009, each entitled “USE OF AEROSOLIZED LEVOFLOXACIN FOR TREATING CHRONIC OBSTRUCTIVE PULMONARY DISEASE,” which are hereby incorporated by reference in their entireties.

### FIELD OF THE INVENTION

The present invention relates to methods and compositions for evaluating aerosolized test compounds. In particular, methods and compositions relating to the use of riboflavin 5′-phosphate are provided.

### BACKGROUND

Various therapeutic compounds have been administered orally or parenterally, e.g. by intravenous, intramuscular or subcutaneous injection. Injection of a drug can be effective, but is often characterized by patient discomfort and inconvenience, and thus poor patient compliance. As a result, it is often considered desirable to provide a therapeutic compound in an oral formulation, as an alternative to, or substitute for, injection. However, oral formulations are often characterized by poor absorption, rapid first-pass metabolism in the liver, slow attainment of effective blood plasma levels and other problems. In addition, oral and parenteral administration typically delivers drugs systemically. In cases where drug is needed only at a local site of disease, systemic administration can result in adverse side effects that could be avoided if the drug was administered locally.

Aerosolized formulations have been used to administer some therapeutic compounds directly to the nasal, sinus, respiratory tract and pulmonary compartments through intra-nasal or oral inhalation. Aerosol administration has several advantages, for example, it can enable high concentration drug delivery to a site with decreased risk of extra-respiratory toxicity associated with non-respiratory routes of drug delivery. There is a continuing need to evaluate the efficacy of particular compounds and formulations before they may be administered through intra-nasal or oral inhalation.

### SUMMARY

The present invention relates to methods and compositions for evaluating aerosolized test compounds. In particular, methods and compositions relating to the use of riboflavin 5′-phosphate are provided.

Some embodiments of the present invention include methods for evaluating a test compound. Some such methods include administering to a first population of individuals a test compound via inhalation of an aerosol; administering to a second population of individuals a placebo comprising riboflavin 5′-phosphate via inhalation of an aerosol; and comparing a biological marker in at least one individual administered the test compound to a biological marker in at least one individual administered the placebo.

In some embodiments, the administering the test compound comprises delivering an aerosolized solution of the test compound.

In some embodiments, the administering is intrapulmonary or intranasal.

In some embodiments, the test compound or placebo are delivered with a pulmonary delivery device. In some such embodiments, the pulmonary delivery device is a nebulizer or a metered dose inhaler.

In some embodiments, the biological marker includes a marker associated with a therapeutic effect, a marker associated with an adverse effect, a marker associated with a toxic effect, a marker associated with a pharmacodynamic parameter.

In some embodiments, the test compound comprises at least one member of the group consisting of antibiotics, antiallergics, anticancer agents, antifungals, antineoplastic agents, analgesics, bronchodilators, antihistamines, antiviral agents, antitussives, anginal preparations, anti-inflammatories, immunomodulators, 5-lipoxygenase inhibitors, leukotriene antagonists, phospholipase A2 inhibitors, phosphodiesterase IV inhibitors, peptides, proteins, steroids, and vaccine preparations.

In some embodiments, the placebo comprises a solution of riboflavin 5′-phosphate. In some such embodiments, the solution comprises a concentration of riboflavin 5′-phosphate greater than about 0.1 mg/ml, greater than about 0.001 mg/L, greater than about 0.005 mg/L, greater than about 0.02 mg/L, and greater than about 0.06 mg/L.

In some embodiments, the riboflavin 5′-phosphate aerosol comprises a respirable delivered dose greater than about 0.001 mg/kg/day, greater than about 0.01 mg/kg/day, greater than about 0.1 mg/kg/day, and greater than about 0.2 mg/kg/day.

In some embodiments, the riboflavin 5′-phosphate aerosol comprises a dose greater than about 0.01 mg/kg/day, greater than about 0.1 mg/kg/day, greater than about 1.0 mg/kg/day, greater than about 2.0 mg/kg/day.

In some embodiments, the aerosolized riboflavin 5′-phosphate comprises a mass median aerodynamic diameter from about 0.5 μm to about 4.5 μm with a geometric standard deviation less than or equal to 3.0 μm.

In some embodiments, the aerosolized riboflavin 5′-phosphate comprises a mass median aerodynamic diameter from about 1.0 μm to about 3.5 μm with a geometric standard deviation less than or equal to 2.7 μm.

In some embodiments, the aerosolized riboflavin 5′-phosphate comprises a mass median aerodynamic diameter from about 1.1 μm to about 3.1 μm with a geometric standard deviation less than or equal to 2.4 μm.

In some embodiments, the placebo is administered at least daily.

In some embodiments, the individuals are animals. In some such embodiments, the animals are mammals. In some such embodiments, the mammals include rat, dog, and human.

Some embodiments of the present invention also include methods for evaluating a test compound comprising: conducting a drug trial of a test compound and a placebo in a population of individuals, wherein the placebo comprises aerosolized riboflavin 5′-phosphate.

In addition to the foregoing, some embodiments of the present invention include aerosols comprising riboflavin 5′-phosphate. In some embodiments, the aerosol includes a solution of riboflavin 5′-phosphate.

In some embodiments, the solution comprises a concentration of riboflavin 5′-phosphate greater than about 0.1 mg/ml, greater than about 0.001 mg/L, greater than about 0.005 mg/L, greater than about 0.02 mg/L, and greater than about 0.06 mg/L.

In some embodiments, the aerosol includes a respirable delivered dose of riboflavin 5′-phosphate greater than about 0.001 mg/kg/day, greater than about 0.01 mg/kg/day, greater than about 0.1 mg/kg/day, and greater than about 0.2 mg/kg/day.

In some embodiments, the aerosol includes a dose of riboflavin 5′-phosphate greater than about 0.01 mg/kg/day, greater than about 0.1 mg/kg/day, greater than about 1.0 mg/kg/day, and greater than about 2.0 mg/kg/day.

In some embodiments, the aerosol includes aerosolized riboflavin 5′-phosphate comprising a mass median aerodynamic diameter from about 0.5 μm to about 4.5 μm with a geometric standard deviation less than or equal to 3.0 μm, a mass median aerodynamic diameter from about 1.0 μm to about 3.5 μm with a geometric standard deviation less than or equal to 2.7 μm, or a mass median aerodynamic diameter from about 1.1 μm to about 3.1 μm with a geometric standard deviation less than or equal to 2.4 μm.

### BRIEF DESCRIPTION OF THE DRAWINGS

FIG. 1 shows graphs for mean riboflavin plasma concentration-time profiles in male or female rats following aerosolized riboflavin 5′-phosphate for 28 days.

FIG. 2A shows graphs for mean riboflavin Cₘₐₓ and AUC_{(0-T)} in male rats following aerosolized doses of riboflavin 5′-phosphate for 28 days. FIG. 2B shows graphs for mean riboflavin Cₘₐₓ and AUC_{(0-T)} in female rats following aerosolized doses of riboflavin 5′-phosphate for 28 days.

FIG. 3 shows graphs of mean riboflavin plasma concentration-time profiles in male and female dogs following aerosolized doses of riboflavin 5′-phosphate for 28 Days.

FIG. 4A and FIG. 4B show graphs for mean riboflavin Cₘₐₓ and AUC_{(0-T)} in dogs following aerosolized doses of riboflavin 5′-phosphate for 28 days.

### DETAILED DESCRIPTION

The present invention relates to methods and compositions for evaluating aerosolized test compounds. In particular, methods and compositions relating to the use of riboflavin 5′-phosphate are provided. Some embodiments include methods for evaluating a test compound that include administering to a population of individuals a test compound or a placebo, in which the placebo includes an aerosolized solution of riboflavin 5′-phosphate, and comparing a biological marker in at least one individual administered the test compound to a biological marker in at least one individual administered the placebo. More embodiments include methods for evaluating a test compound that include conducting a drug trial of a test compound and a placebo in a population of individuals, in which the placebo comprises an aerosolized solution of riboflavin 5′-phosphate.

Applicant has discovered that administration of aerosolized solutions of riboflavin 5′-phosphate by inhalation is well tolerated, with no adverse clinical observations detected, and no indications of local or systemic toxicity in model mammalian systems. For example, in dogs, no adverse effects were observed after oronasal administration of riboflavin 5′-phosphate for 28 consecutive days at 1.34 mg/kg/day. In rats, no adverse effects were observed after nose-only aerosol administration of riboflavin 5′-phosphate at 2.4 mg/kg/day for 28 consecutive days. The absence of adverse clinical observations and no indications of local or systemic toxicity make aerosolized solutions of riboflavin 5′-phosphate a good candidate as a placebo control in methods for evaluating test compounds. Thus, some embodiments of the present invention provide methods for evaluating test compounds using aerosolized solutions of riboflavin 5′-phosphate as a control.

Some methods to evaluate test compounds can include clinical trials. Clinical trials are conducted to gather safety and efficacy data, for example, on new therapeutic compounds, new formulations, and new uses of therapeutic compounds. A clinical trial can include a randomized controlled study which may be designed to be randomized, blind, and/or placebo-controlled. In a randomized study, each study subject may be randomly assigned to receive the study treatment or a placebo. In a blind study, a subject involved in the study may not know whether they receive the study treatment or placebo, and if the study is double-blind, the researcher also may not know which treatment is being given to any given subject. And, in a placebo-controlled study, a group of subjects may receive a study treatment, and a separate control group of subjects receive a placebo.

One purpose of a placebo group is to account for the placebo effect, that is, effects from treatment that do not depend on the treatment itself. Such factors include knowing one is receiving a treatment, attention from health care professionals, and the expectations of a treatment's effectiveness by those running the research study. Without a placebo group to compare against, it may not possible to know whether the treatment itself had any effect. Therefore, the use of placebos is a standard control component of most clinical trials which attempt to make some sort of quantitative assessment of the efficacy of therapeutic compounds and/or treatments.

Some embodiments include the use of aerosolized solutions of riboflavin 5′-phosphate as a placebo. In some such embodiments, a test compound or placebo can be administered to a population of individuals. The effect of the test compound can be observed by comparing a biological marker in at least one individual administered the test compound to a biological marker in at least one individual administered the placebo.

Examples of biological markers include markers associated with therapeutic effects, markers associated with adverse effects, markers associated with toxic effects, and markers associated with pharmacodynamic parameters. Generally, therapeutic effects are desirable and/or beneficial, while adverse effects are harmful and/or undesirable. Both types of effect may include physiological or behavioral changes in an individual. Pharmacodynamic parameters are associated with the physiological effects of a test compound on the body of an individual. Pharmacokinetic parameters are associated with the effect of a body on a test compound.

As used herein “individual” includes an animal. The term “animal” is used in its ordinary and broadest sense and includes invertebrates, for example, mammals, primates, rodents, rats, dogs and humans.

Some embodiments utilize riboflavin 5′-phosphate or salts thereof. Riboflavin 5′-phosphate may also be known as flavin mononucleotide and vitamin B₂ phosphate. Riboflavin 5′-phosphate has the following structure:

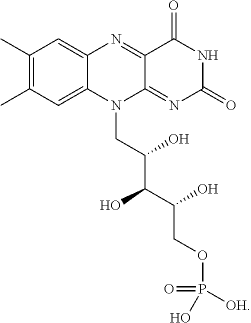

Generally, in some methods to evaluate test compounds, a placebo can include a formulation that is substantially similar to the formulation of the test compound. It will be understood that the placebo formulation does not typically contain a test compound. Conversely, a formulation containing a test compound does not typically contain a placebo. Formulations of test compounds and placebos will vary according to the mode of administration as well as according to the properties of the test compound or placebo. Examples of formulations for aerosol delivery of compounds are disclosed in U.S. Patent Application Publication No. 2006-0276483, incorporated by reference in its entirety.

Some solutions of riboflavin 5′-phosphate may have a yellow/orange/red color. As will be understood, the color of a riboflavin 5′-phosphate solution can vary according to factors such as the concentration of the riboflavin 5′-phosphate solution. In some embodiments, the color of a riboflavin 5′-phosphate solution can advantageously mask the color of a solution containing a test compound. In some such embodiments, a formulation containing a test compound may also contain riboflavin 5′-phosphate. Thus, solutions containing the test compound may be color matched with placebo solutions lacking the test compound.

Some methods for evaluating test compounds can include, for example, a solution of riboflavin 5′-phosphate comprising a concentration greater than about 0.001 mg/ml, about 0.002 mg/ml, about 0.003 mg/ml, about 0.004 mg/ml, about 0.005 mg/ml, about 0.006 mg/ml, about 0.007 mg/ml, about 0.008 mg/ml, about 0.009 mg/ml, and about 0.01 mg/ml, about 0.02 mg/ml, about 0.03 mg/ml, about 0.04 mg/ml, about 0.05 mg/ml, about 0.06 mg/ml, about 0.07 mg/ml, about 0.08 mg/ml, about 0.09 mg/ml, and about 0.1 mg/ml, about 0.2 mg/ml, about 0.3 mg/ml, about 0.4 mg/ml, about 0.5 mg/ml, about 0.6 mg/ml, about 0.7 mg/ml, about 0.8 mg/ml, about 0.9 mg/ml, and about 1 mg/ml, about 2 mg/ml, about 3 mg/ml, about 4 mg/ml, about 5 mg/ml, about 6 mg/ml, about 7 mg/ml, about 8 mg/ml, about 9 mg/ml, and about 10 mg/ml.

Some methods for evaluating test compounds can include a respirable delivered dose of riboflavin 5′-phosphate greater than about 0.001 mg/kg/day, about 0.002 mg/kg/day, about 0.003 mg/kg/day, about 0.004 mg/kg/day, about 0.005 mg/kg/day, about 0.006 mg/kg/day, about 0.007 mg/kg/day, about 0.008 mg/kg/day, about 0.009 mg/kg/day, and about 0.01 mg/kg/day, about 0.02 mg/kg/day, about 0.03 mg/kg/day, about 0.04 mg/kg/day, about 0.05 mg/kg/day, about 0.06 mg/kg/day, about 0.07 mg/kg/day, about 0.08 mg/kg/day, about 0.09 mg/kg/day, and about 0.1 mg/kg/day, about 0.2 mg/kg/day, about 0.3 mg/kg/day, about 0.4 mg/kg/day, about 0.5 mg/kg/day, about 0.6 mg/kg/day, about 0.7 mg/kg/day, about 0.8 mg/kg/day, about 0.9 mg/kg/day, and about 1 mg/kg/day, about 2 mg/kg/day, about 3 mg/kg/day, about 4 mg/kg/day, about 5 mg/kg/day, about 6 mg/kg/day, about 7 mg/kg/day, about 8 mg/kg/day, about 9 mg/kg/day, and about 10 mg/kg/day.

Some methods for evaluating test compounds can include a dose of riboflavin 5′-phosphate greater than about 0.001 mg/kg/day, about 0.002 mg/kg/day, about 0.003 mg/kg/day, about 0.004 mg/kg/day, about 0.005 mg/kg/day, about 0.006 mg/kg/day, about 0.007 mg/kg/day, about 0.008 mg/kg/day, about 0.009 mg/kg/day, and about 0.01 mg/kg/day, about 0.02 mg/kg/day, about 0.03 mg/kg/day, about 0.04 mg/kg/day, about 0.05 mg/kg/day, about 0.06 mg/kg/day, about 0.07 mg/kg/day, about 0.08 mg/kg/day, about 0.09 mg/kg/day, and about 0.1 mg/kg/day, about 0.2 mg/kg/day, about 0.3 mg/kg/day, about 0.4 mg/kg/day, about 0.5 mg/kg/day, about 0.6 mg/kg/day, about 0.7 mg/kg/day, about 0.8 mg/kg/day, about 0.9 mg/kg/day, and about 1 mg/kg/day, about 2 mg/kg/day, about 3 mg/kg/day, about 4 mg/kg/day, about 5 mg/kg/day, about 6 mg/kg/day, about 7 mg/kg/day, about 8 mg/kg/day, about 9 mg/kg/day, and about 10 mg/kg/day.

Some methods for evaluating test compounds can include aerosolized riboflavin 5′-phosphate comprises a mass median aerodynamic diameter from about 0.5 μm to about 4.5 μm with a geometric standard deviation less than or equal to 3.0 μm. More methods for evaluating test compounds can include aerosolized riboflavin 5′-phosphate comprises a mass median aerodynamic diameter from about 1.0 μm to about 3.5 μm with a geometric standard deviation less than or equal to 2.7 μm. More methods for evaluating test compounds can include aerosolized riboflavin 5′-phosphate comprises a mass median aerodynamic diameter from about 1.1 μm to about 3.1 μm with a geometric standard deviation less than or equal to 2.4 μm.

### Modes of Administration

Test compounds and placebos can be administered by various modes of delivery, including pulmonary and nasal modes of delivery.

### Pulmonary Administration

Some embodiments can employ pulmonary delivery of test compounds or placebos. The test compound or placebo is delivered to the lungs while inhaling and traverses across the lung epithelial lining to the blood stream. A wide range of mechanical devices designed for pulmonary delivery of therapeutic products can be employed, including but not limited to nebulizers, metered dose inhalers, and powder inhalers, all of which are familiar to those skilled in the art. These devices employ formulations suitable for the dispensing of test compound or placebo. Typically, each formulation is specific to the type of device employed and can involve the use of an appropriate propellant material, in addition to diluents, adjuvants, and/or carriers useful in therapy.

Generally, inhaled particles are subject to deposition by one of two mechanisms: impaction, which usually predominates for larger particles, and sedimentation, which is prevalent for smaller particles. Impaction occurs when the momentum of an inhaled particle is large enough that the particle does not follow the air stream and encounters a physiological surface. In contrast, sedimentation occurs primarily in the deep lung when very small particles which have traveled with the inhaled air stream encounter physiological surfaces as a result of random diffusion within the air stream.

For pulmonary administration, the upper airways are avoided in favor of the middle and lower airways. Pulmonary drug delivery may be accomplished by inhalation of an aerosol through the mouth and throat. Particles having a mass median aerodynamic diameter (MMAD) of greater than about 5 μm generally do not reach the lung; instead, they tend to impact the back of the throat and are swallowed and possibly orally absorbed. Particles having diameters of about 2 to about 5 μm are small enough to reach the upper- to mid-pulmonary region (conducting airways), but are too large to reach the alveoli. Smaller particles, i.e., about 0.5 to about 2 μm, are capable of reaching the alveolar region. Particles having diameters smaller than about 0.5 μm can also be deposited in the alveolar region by sedimentation, although very small particles may be exhaled. Measures of particle size can be referred to as volumetric mean diameter (VMD), mass median diameter (MMD), or MMAD. These measurements may be made by impaction (MMD and MMAD) or by laser (VMD). For liquid particles, VMD, MMD and MMAD may be the same if environmental conditions are maintained, e.g. standard humidity. However, if humidity is not maintained, MMD and MMAD determinations will be smaller than VMD due to dehydration during impator measurements. For the purposes of this description, VMD, MMD and MMAD measurements are considered to be under standard conditions such that descriptions of VMD, MMD and MMAD will be comparable. Similarly, dry powder particle size determinations in MMD, and MMAD are also considered comparable.

Aerosol particle size may be expressed in terms of the mass median aerodynamic diameter (MMAD). Large particles (e.g., MMAD >5 μm) may deposit in the upper airway because they are too large to navigate the curvature of the upper airway. Small particles (e.g., MMAD <2 μm) may be poorly deposited in the lower airways and thus become exhaled, providing additional opportunity for upper airway deposition. Hence, intolerability (e.g., cough and bronchospasm) may occur from upper airway deposition from both inhalation impaction of large particles and settling of small particles during repeated inhalation and expiration. Thus, in one embodiment, an optimum particle size is used (e.g., MMAD=2-5 μm) in order to maximize deposition at a mid-lung site and to minimize intoleratiblity associated with upper airway deposition. Moreover, generation of a defined particle size with limited geometric standard deviation (GSD) may optimize deposition and tolerability. Narrow GSD limits the number of particles outside the desired MMAD size range. In one embodiment, an aerosol containing one or more compounds disclosed herein is provided having a MMAD from about 2 μm to about 5 μm with a GSD of less than or equal to about 2.5 μm. In another embodiment, an aerosol having an MMAD from about 2.8 μm to about 4.3 μm with a GSD less than or equal to 2 μm is provided. In another embodiment, an aerosol having an MMAD from about 2.5 μm to about 4.5 μm with a GSD less than or equal to 1.8 μm is provided.

The test compound or placebo and/or other optional active ingredients are advantageously prepared for pulmonary delivery in particulate form with an average particle size of from 0.1 μm or less to 10 μm or more, more preferably from about 0.2, 0.3, 0.4, 0.5, 0.6, 0.7, 0.8, or 0.9 μm to about 1.0, 1.5, 2.0, 2.5, 3.0, 3.5, 4.0, 4.5, 5.0, 5.5, 6.0, 6.5, 7.0, 7.5, 8.0, 8.5, 9.0, or 9.5 μm. Pharmaceutically acceptable carriers for pulmonary delivery of test compound or placebo include carbohydrates such as trehalose, mannitol, xylitol, sucrose, lactose, and sorbitol. Other ingredients for use in formulations can include DPPC, DOPE, DSPC, and DOPC. Natural or synthetic surfactants can be used, including polyethylene glycol and dextrans, such as cyclodextran. Bile salts and other related enhancers, as well as cellulose and cellulose derivatives, and amino acids can also be used. Liposomes, microcapsules, microspheres, inclusion complexes, and other types of carriers can also be employed.

In one embodiment, a nebulizer is selected on the basis of allowing the formation of an aerosol of a test compound or placebo disclosed herein having an MMAD predominantly between about 2 to about 5 μm. For aqueous and other non-pressurized liquid systems, a variety of nebulizers (including small volume nebulizers) are available to aerosolize the formulations. Compressor-driven nebulizers incorporate jet technology and use compressed air to generate the liquid aerosol. Such devices are commercially available from, for example, Healthdyne Technologies, Inc.; Invacare, Inc.; Mountain Medical Equipment, Inc.; Pari Respiratory, Inc.; Mada Medical, Inc.; Puritan-Bennet; Schuco, Inc., DeVilbiss Health Care, Inc.; and Hospitak, Inc. Ultrasonic nebulizers rely on mechanical energy in the form of vibration of a piezoelectric crystal to generate respirable liquid droplets and are commercially available from, for example, Omron Heathcare, Inc. and DeVilbiss Health Care, Inc. Vibrating mesh nebulizers rely upon either piezoelectric or mechanical pulses to respirable liquid droplets generate. Other examples of nebulizers for use with test compounds or placebos described herein are described in U.S. Pat. Nos. 4,268,460; 4,253,468; 4,046,146; 3,826,255; 4,649,911; 4,510,929; 4,624,251; 5,164,740; 5,586,550; 5,758,637; 6,644,304; 6,338,443; 5,906,202; 5,934,272; 5,960,792; 5,971,951; 6,070,575; 6,192,876; 6,230,706; 6,349,719; 6,367,470; 6,543,442; 6,584,971; 6,601,581; 4,263,907; 5,709,202; 5,823,179; 6,192,876; 6,644,304; 5,549,102; 6,083,922; 6,161,536; 6,264,922; 6,557,549; and 6,612,303 all of which are hereby incorporated by reference in their entirety. Commercial examples of nebulizers that can be used with the fluoroquinolone antimicrobial agents described herein include Respirgard II®, Aeroneb®, Aeroneb® Pro, and Aeroneb® Go produced by Aerogen; AERx® and AERx Essence™ produced by Aradigm; Porta-Neb®, Freeway Freedom™, Sidestream, Ventstream and I-neb produced by Respironics, Inc.; and PARI LC-Plus®, PARI LC-Star®, and e-Flow™ produced by PARI, GmbH. By further non-limiting example, U.S. Pat. No. 6,196,219, is hereby incorporated by reference in its entirety. Further methods for utilizing nebulizers are disclosed in U.S. Patent Application Publication No. 2006-0276483, incorporated by reference in its entirety.

Pharmaceutical formulations suitable for use with a nebulizer, either jet or ultrasonic, typically comprise a test compound or placebo dissolved or suspended in water at a concentration of about 0.01 or less to 100 mg or more of inhibitor per mL of solution, preferably from about 0.1, 1, 2, 3, 4, 5, 6, 7, 8, 9, or 10 mg to about 15, 20, 25, 30, 35, 40, 45, 50, 55, 60, 65, 70, 75, 80, 85, or 90 mg per mL of solution. The formulation can also include a buffer and a simple sugar (e.g., for protein stabilization and regulation of osmotic pressure). The nebulizer formulation can also contain a surfactant, to reduce or prevent surface induced aggregation of the test compound or placebo caused by atomization of the solution in forming the aerosol.

Some embodiments utilize a meter dose inhaler (MDI). A propellant driven inhaler (pMDI) releases a metered dose of test compound or placebo upon each actuation. The test compound or placebo is formulated as a suspension or solution of the test compound or placebo in a suitable propellant such as a halogenated hydrocarbon. pMDIs are described in, for example, Newman, S. P., Aerosols and the Lung, Clarke et al., eds., pp. 197-224 (Butterworths, London, England, 1984).

In some embodiments, the particle size of the test compound or placebo in an MDI may be optimally chosen. In some embodiments, the particles of active ingredient have diameters of less than about 50 μm. In some embodiments, the particles have diameters of less than about 10 μm. In some embodiments, the particles have diameters of from about 1 μm to about 5 μm. In some embodiments, the particles have diameters of less than about 1 μm. In one advantageous embodiment, the particles have diameters of from about 2 μm to about 5 μm.

Formulations for use with a metered-dose inhaler device generally comprise a finely divided powder containing the active ingredients suspended in a propellant with the aid of a surfactant. The propellant can include conventional propellants, such as chlorofluorocarbons, hydrochlorofluorocarbons, hydrofluorocarbons, and hydrocarbons. Preferred propellants include trichlorofluoromethane, dichlorodifluoromethane, dichlorotetrafluoroethanol, 1,1,1,2-tetrafluoroethane, and combinations thereof. Examples of medicinal aerosol preparations containing hydrofluoroalkanes are presented in U.S. Pat. No. 6,585,958; U.S. Pat. No. 2,868,691; and U.S. Pat. No. 3,014,844, all of which are hereby incorporated by reference in their entirety. Suitable surfactants include sorbitan trioleate, soya lecithin, and oleic acid.

Some embodiments utilize dry powder inhalers. There are two major designs of dry powder inhalers. One design is the metering device in which a reservoir for the test compound or placebo is placed within the device and a dose of the test compound or placebo is placed into the inhalation chamber. The second is a factory-metered device in which each individual dose has been manufactured in a separate container. Both systems depend upon the formulation of test compound or placebo into small particles of mass median diameters from about 1 to about 5 μm, and usually involve co-formulation with larger excipient particles (typically 100 μm diameter lactose particles). Test compound or placebo powder is placed into the inhalation chamber (either by device metering or by breakage of a factory-metered dosage) and the inspiratory flow of the individual accelerates the powder out of the device and into the oral cavity. Non-laminar flow characteristics of the powder path cause the excipient-test compound or placebo aggregates to decompose, and the mass of the large excipient particles causes their impaction at the back of the throat, while the smaller test compound or placebo particles are deposited deep in the lungs.

As with liquid nebulization and MDIs, particle size of the test compound or placebo aerosol formulation may be optimized. If the particle size is larger than about 5 μm MMAD then the particles are deposited in upper airways. If the particle size of the aerosol is smaller than about 1 μm then it is delivered into the alveoli and may get transferred into the systemic blood circulation.

Additional examples of dry powder inhalers for use herein are described in U.S. Pat. Nos. 4,811,731; 5,113,855; 5,840,279; 3,507,277; 3,669,113; 3,635,219; 3,991,761; 4,353,365; 4,889,144, 4,907,538; 5,829,434; 6,681,768; 6,561,186; 5,918,594; 6,003,512; 5,775,320; 5,740,794; and 6,626,173, all of which are hereby incorporated by reference in their entirety.

Formulations for dispensing from a powder inhaler device typically comprise a finely divided dry powder containing test compound or placebo, optionally including a bulking agent, such as lactose, sorbitol, sucrose, mannitol, trehalose, or xylitol in an amount that facilitates dispersal of the powder from the device, typically from about 1 wt. % or less to 99 wt. % or more of the formulation, preferably from about 5, 10, 15, 20, 25, 30, 35, 40, 45, or 50 wt. % to about 55, 60, 65, 70, 75, 80, 85, or 90 wt. % of the formulation.

Some embodiments include aerosols comprising riboflavin 5′-phosphate. Aerosols include suspensions of solid particles and suspensions of liquids in air. In some embodiments, the aerosol includes a solution of riboflavin 5′-phosphate.

In some embodiments, the solution of riboflavin 5′-phosphate comprises a concentration greater than about 0.1 mg/ml, greater than about 0.001 mg/L, greater than about 0.005 mg/L, greater than about 0.02 mg/L, and greater than about 0.06 mg/L.

In some embodiments, the aerosol includes a respirable delivered dose of riboflavin 5′-phosphate greater than about 0.001 mg/kg/day, greater than about 0.01 mg/kg/day, greater than about 0.1 mg/kg/day, and greater than about 0.2 mg/kg/day.

In some embodiments, the aerosol includes a dose of riboflavin 5′-phosphate greater than about 0.01 mg/kg/day, greater than about 0.1 mg/kg/day, greater than about 1.0 mg/kg/day, and greater than about 2.0 mg/kg/day.

In some embodiments, the aerosol includes aerosolized riboflavin 5′-phosphate comprising a mass median aerodynamic diameter from about 0.5 μm to about 4.5 μm with a geometric standard deviation less than or equal to 3.0 μm, a mass median aerodynamic diameter from about 1.0 μm to about 3.5 μm with a geometric standard deviation less than or equal to 2.7 μm, or a mass median aerodynamic diameter from about 1.1 μm to about 3.1 μm with a geometric standard deviation less than or equal to 2.4 μm.

More methods for administering aerosols are disclosed in U.S. Patent Application Publication No. 2006-0276483, incorporated by reference in its entirety.

### Nasal Administration

Nasal delivery allows the passage of a placebo and/or test compound to the blood stream directly after administering the therapeutic product to the nose, without the necessity for deposition of the product in the lung. In some embodiments, test compounds and placebos can be administered as a nasal spray or nasal drop. Nasal sprays may be liquid or solid nasal sprays. The nasal sprays may be aerosol or non-aerosol nasal sprays. Nasal delivery systems can include: 1) aerosolized metered dose pumps, 2) manual metered dose pumps, and 3) metered dose spray-producing squeeze bottles. Each of these is effective in providing for the rapid absorption of test compounds into the blood stream of a subject.

An aerosol may be insufflated using a suitable mechanical apparatus. In some embodiments, the apparatus may include a reservoir and sprayer, which is a device adapted to expel the pharmaceutical dose in the form of a spray. A number of doses of the test compound or placebo to be administered may be contained within the reservoir, optionally in a liquid solution or suspension or in a solid particulate formulation, such as a solid particulate mixture.

In some embodiments, the apparatus is a pump sprayer that includes a metering pump. In some embodiments, the apparatus includes a pressurized spray device, in which the sprayer includes a metering valve and the putative pharmaceutical composition further comprises a pharmaceutically acceptable propellant. Exemplary propellants are disclosed herein, and include one or mixture of chlorofluorocarbons, such as dichlorodifluoromethane, as well as hydrofluorocarbons, such as 1,1,1,2-tetrafluoroethane, and 1,1,1,2,3,3,3-heptafluoropropane. Suitable pressurized spray devices are well known and will be familiar to those of skill in the art.

In some embodiments, powders can be administered using a nasal insufflator. In some embodiments, powders may be contained within a capsule, which is inserted into an insufflation device. The capsule is punctured by a needle, which makes apertures at the top and bottom of the capsule. Air or other pharmaceutically acceptable propellant is then sent through the needle to blow out powder particles. In some embodiments, pharmaceutically acceptable propellants include, for example, ethyl chloride, butane, propane, dichlorodifluoromethane, dichlorotetrafluoroethane, and trichloromonofluoromethane.

Some test compounds may be so slightly soluble in water that a putative therapeutically effective amount cannot be dissolved in a volume of aqueous solvent that is amenable to nasal insufflation as an aerosol or non-aerosol spray. The volume of insufflate that is suitable for nasal administration will vary with the nature of the test compounds to be evaluated. In some embodiments, volume of insufflate that is suitable for nasal administration can be in the range of about 25 μl to about 250 μl per nostril, preferably about 50 μl to about 150 μl per nostril, and particularly about 50 μl to about 100 μl per nostril. The solid or liquid particles may be suspended in an air stream by the action of a micronizing pump, a stream of aerosolizing inert gas, etc.

### Test Compounds

Test compounds can include compounds evaluated for a therapeutic effect in an individual. As used herein, the term “test compound” is used in its ordinary and broadest meaning and includes substances that may be useful in the diagnosis, cure, mitigation, treatment or prevention of disease, or to affect the structure or function of the body. Examples of test compounds can include compounds that may be useful as antibiotics, antiallergics, anticancer agents, antifungals, antineoplastic agents, analgesics, bronchodilators, antihistamines, antiviral agents, antitussives, anginal preparations, anti-inflammatories, immunomodulators, 5-lipoxygenase inhibitors, leukotriene antagonists, phospholipase A₂ inhibitors, phosphodiesterase IV inhibitors, peptides, proteins, steroids, and vaccine preparations.

Preferably, a test compound is suitable for oral and/or nasal inhalation. In some embodiments, a test compound is present in a formulation adapted for aerosol administration. Examples of excipients are disclosed herein, and include cosolvents (e.g., ethanol, water), surfactants (e.g., oleic acid, sorbitan esters, polyoxyethylenes, glycols, oligolactic acids) and others known to those skilled in the art.

### Therapeutic Use of Riboflavin 5′-Phosphate

Some embodiments include the therapeutic use of aerosolized riboflavin 5′-phosphate. In some such embodiments riboflavin 5′-phosphate can be administered to a subject in need thereof by any method described herein. For example, in some embodiments a riboflavin 5′-phosphate solution can be administered to a subject as an aerosolized solution. Such methods allow for fast delivery and absorption of riboflavin. Subjects include mammals, for example humans. Dosage can be determined empirically. Riboflavin 5′-phosphate can be used to treat a variety of indications.

In some such embodiments, riboflavin 5′-phosphate can be used with beta blockers to treat or prevent migraine headaches. A randomized placebo-controlled trial examined the effect of 400 mg of riboflavin/day for three months on migraine prevention in 54 men and women with a history of recurrent migraine headaches (Schoenen J, et al. Effectiveness of high-dose riboflavin in migraine prophylaxis. A randomized controlled trial. Neurology. 1998; 50(2):466-470). Riboflavin was significantly better than placebo in reducing attack frequency and the number of headache days, though the beneficial effect was most pronounced during the third month of treatment. A more recent study by the same investigators found that treatment with either a medication called a beta-blocker or high-dose riboflavin resulted in clinical improvement, but each therapy appeared to act on a distinct pathological mechanism: beta-blockers on abnormal cortical information processing and riboflavin on decreased brain mitochondrial energy reserve (Sandor P S, et al. Prophylactic treatment of migraine with beta-blockers and riboflavin: differential effects on the intensity dependence of auditory evoked cortical potentials. Headache. 2000; 40(1):30-35). A small study in 23 patients reported a reduction in median migraine attack frequency after supplementation with 400 mg of riboflavin daily for three months (Boehnke C, et al. High-dose riboflavin treatment is efficacious in migraine prophylaxis: an open study in a tertiary care centre. Eur J. Neurol. 2004; 11(7):475-477).

In some embodiments, riboflavin 5′-phosphate can be used to treat neonatal jaundice. For example, riboflavin 5′-phosphate can be used as part of phototherapy treatment of neonatal jaundice. The light used to irradiate the infants breaks down not only bilirubin, the toxin causing the jaundice, but the naturally occurring riboflavin within the infant's blood as well, so that extra supplementation is necessary.

In some embodiments, riboflavin 5′-phosphate can be used to treat riboflavin deficiency. For example, riboflavin 5′-phosphate is beneficial in patients with riboflavin deficiency (e.g., ariboflavinosis). Ariboflavinosis may cause weakness, throat swelling/soreness, glossitis (tongue swelling), angular stomatitis/cheilosis (skin cracking or sores at the corners of the mouth), dermatitis (skin irritation), or anemia. Particular groups may be especially susceptible to riboflavin deficiency, including the elderly, those with chronic illnesses, the poor, and those with alcohol dependency.

In some embodiments, riboflavin 5′-phosphate can be used to treat iron deficiency anemia and sickle cell anemia. In iron deficiency anemia and sickle cell anemia levels of riboflavin may be low. Correction of riboflavin deficiency in individuals who are both riboflavin deficient and iron deficient can improve response to iron therapy.

In some embodiments, riboflavin 5′-phosphate can be used to enhance cognitive function. Adequate nutrient supplementation with riboflavin may be required for the maintenance of adequate cognitive function. Treatment with B-vitamins including riboflavin has been reported to improve scores of depression and cognitive function in patients taking tricyclic antidepressants. This may be related to tricyclic-caused depletion of riboflavin levels.

In some embodiments, riboflavin 5′-phosphate can be used to treat depression. Adequate nutrient supplementation with riboflavin may be required for the maintenance of adequate cognitive function. Treatment with B-vitamins, including riboflavin, has been reported to improve depression scores in patients taking tricyclic antidepressants. This may be related to tricyclic-caused depletion of riboflavin levels.

In some embodiments, riboflavin 5′-phosphate can be used to treat preeclampsia. Preeclampsia is defined as the presence of elevated blood pressure, protein in the urine, and edema (significant swelling) during pregnancy. About 5% of women with preeclampsia may progress to eclampsia, a significant cause of maternal death. Eclampsia is characterized by seizures, in addition to high blood pressure and increased risk of hemorrhage (severe bleeding) (Crombleholme W R. Obstetrics. In: Tierney L M, McPhee S J, Papadakis M A, eds. Current Medical Treatment and Diagnosis. 37th ed. Stamford: Appleton and Lange; 1998:731-734). A study in 154 pregnant women at increased risk of preeclampsia found that those who were riboflavin deficient were 4.7 times more likely to develop preeclampsia than those who had adequate riboflavin nutritional status. Decreased intracellular levels of flavocoenzymes could cause mitochondrial dysfunction, increase oxidative stress, and interfere with nitric oxide release and thus blood vessel dilation—all of these changes have been associated with preeclampsia (Wacker J, et al. Riboflavin deficiency and preeclampsia. Obstet. Gynecol. 2000; 96(1):38-44).

### EXAMPLES

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All references cited herein are incorporated by reference in their entirety.

### Example 1

### Riboflavin 5′-Phosphate: A 28-Day Aerosolized Liquid Inhalation Toxicity Study in Sprague-Dawley Rats

The objective of the study was to determine the toxicity and toxicokinetic profile of the test article, riboflavin 5′-phosphate, following inhalation (nose-only) administration to rats for 28 consecutive days. Table 1 shows respirable delivered dose for each Group.

TABLE 1

| | | Projected | | | | | |
|---|---|---|---|---|---|---|---|
| | | dose level | | | | | |
| | | of riboflavin | Respirable | Number of animals | | | |
| Group | Group | 5′-phosphate^{a} | delivered dose^{b} | Main study | | Toxicokinetic | |
| number | designation | (mg/kg/day) | (mg/kg/day) | Male | Female | Male | Female |
| 1 | Vehicle | 0 | 0 | 10 | 10 | 3 | 3 |
| | control | | | | | | |
| 2 | Low dose | 0.24 | 0.024 | 10 | 10 | 6 | 6 |
| 3 | Mid dose | 0.96 | 0.096 | 10 | 10 | 6 | 6 |
| 4 | High dose | 2.40 | 0.24 | 10 | 10 | 6 | 6 |

^{a}Projected dose levels were calculated based on an estimated body weight of 0.250 kg.
^{b}FDA assumed deposition of 10%

Test Article: riboflavin 5′-phosphate sodium salt hydrate; alternate identity: vitamin B₂ phosphate; description: yellow crystals; potency: 74%. Vehicle article: saline (0.9% (w/v) NaCl for injection); description: clear colorless solution. Test system: rat (rattus norvegicus); strain: Sprague-Dawley Crl:CD (SD).

### Preparation of Test and Control Articles Formulations

The test (0.5 mg/mL, 2.0 mg/mL, 5.0 mg/mL) and/or vehicle control formulations (0 mg/mL) for each group were prepared fresh daily on each day by dissolving the test article in saline. All prepared test article formulations were protected from light and kept at room temperature (RT).

### Treatment

### Acclimatization to Exposure System

Before the animals were presented with the exposure atmosphere, all animals (including controls) were accustomed to the restraint procedure over 3 days. The animals were gradually accustomed to restraint in the dosing tubes used during the exposures, up to at maximum of 1 hour.

### Aerosol Generation Characteristics and Theoretical Dose Levels

Treatment: Daily inhalation by nose-only exposure for 60 minutes. Duration of treatment: 28 Days. Projected aerosol concentrations and dose levels are shown in Table 2.

TABLE 2

| | | | | Projected | Projected | |
|---|---|---|---|---|---|---|
| | | Projected | Projected | aerosol | formulation | |
| | | dose level | respirable | concentration | concentration | |
| | | of riboflavin | delivered | of riboflavin | of riboflavin | Duration of |
| Group | Group | 5′-phosphate^{a} | dose^{b} | 5′-phosphate | 5′-phosphate | exposure |
| number | designation | (mg/kg/day) | (mg/kg/day) | (mg/L)^{c} | (mg/ml) | (min) |
| 1 | Vehicle | 0 | 0 | 0 | 0 | 60 |
| | control | | | | | |
| 2 | Low dose | 0.24 | 0.024 | 0.006 | 0.5 | 60 |
| 3 | Mid dose | 0.96 | 0.096 | 0.024 | 2.0 | 60 |
| 4 | High dose | 2.40 | 0.24 | 0.060 | 5.0 | 60 |

^{a}Projected dose levels were calculated based on an estimated body weight of 0.250 kg
^{b}FDA assumed deposition of 10%
^{c}Projected aerosol of concentrations were determined based on the results obtained from the technical validation study conducted prior to this study (ITR Study No. 40242)

Estimation of achieved doses

$${D}_{L} = \frac{{E}_{c} \times RMV \times T}{BW}$$

D_{L}=Achieved dose levels (mg/kg/day)
E_{c}=Actual concentration delivered to the animals (mg/L air)
RMV=Respiratory minute volume (L/min), calculated according to the method of Bide, Armour and Yee 2000, as detailed below:
   RMV(L)=0.499×W(kg)^{0.809}
Time, i.e., the duration of exposure (min.)
BW=Mean body weight (kg) during exposure period

This estimation of achieved dose assumed 100% deposition within the respiratory tract (Bide R. W., et al. Allometric Respiration/Body Mass Data for Animals to be Used for Estimates of Inhalation Toxicity to Young Adult Humans. J. App. Toxicol., 2000 Vol. 20, incorporated by reference in its entirety).

### Inhalation Exposure System

The aerosol was produced by metering the flow of the test article or vehicle formulations to 3 clinical nebulizers (Sidestream) connected to high velocity airstreams (10 L/min to each nebuliser). The aerosol produced was discharged through a 40 mm diameter tube into a flow-past inhalation exposure system. The airflow rate through the exposure system was monitored and recorded manually during the aerosol generation. Airflow to the exposure system was controlled by the absolute volume of air supplying the aerosol generators using variable area flow meters.

Control of the aerosol exhaust flow from the animal exposure system was achieved using an exhaust valve, and the overall balance of airflows in the exposure system was monitored using pressure gauges. The system provided a minimum of 1.0 L/min atmosphere to each animal exposure port and was balanced to ensure a slight positive pressure at the site of the animal exposure. This ensured that there was no dilution of the generated aerosol. An equal delivery of aerosol to each proposed exposure position was achieved by employing a distribution network that was identical for each individual exposure position attached to the system.

### Exposure System Monitoring

Determinations of aerosol concentration, particle size distribution, oxygen concentration, relative humidity and temperature was performed on test atmosphere samples collected from a representative port of the exposure system, with a collection sample flow-rate of 1 L/min. The sample flow rates were precisely controlled using variable area flow meters that were calibrated using a primary airflow calibrator before use. The absolute volume of each aerosol concentration sample was measured using a wet type gas meter.

### Determination of Aerosol Concentration

Prior to dosing of the rats, atmosphere homogeneity in the exposure system was tested for Groups 2 to 4 by collecting multiple aerosol samples from the top, middle and bottom tiers of the exposure system. A coefficient of variance <20% between tiers confirmed the homogeneity of the aerosol. During the treatment period, multiple aerosol concentration samples were collected onto filters from all groups, including the control groups on a daily basis, for each aerosol generation occasion. The collected filters were transferred to the analytical chemistry laboratory of ITR for chemical determination of Riboflavin 5′-phosphate concentration using a validated analytical method (ITR Study No. 40241).

### Determination of Particle Size Distribution

The distribution of particle size in the generated aerosols for Groups 2 to 4 was measured weekly during the treatment period by collecting samples into a 7-Stage Mercer Cascade Impactor and the sample substrates obtained were transferred to the analytical chemistry laboratory of ITR Laboratories Canada Inc. for the chemical determination of the particle size of the aerosolized Riboflavin 5′-phosphate using a validated analytical method (ITR Study No. 40241). The distribution of particle size in the generated aerosols for Group 1 was estimated once weekly by gravimetric determination. The mass median aerodynamic diameter (MMAD) and the Geometric Standard Deviation (GSD) were calculated based on the results obtained from the impactor using a log-probit transformation.

### Clinical Evaluations

Assessments of mortality, clinical signs, body weights, food consumption, ophthalmology, functional observation battery and clinical chemistry, hematology, coagulation and urinalysis were performed. Plasma prepared from blood samples were collected from designated animals on the first and last days of the study and analyzed for Riboflavin 5′-phosphate content and toxicokinetic evaluation. All toxicology (main study) animals were euthanized upon completion of the 28-day treatment period and selected tissues were retained, weighed and examined microscopically. Toxicokinetic animals were euthanized following completion of blood sampling and were not subjected to necropsy examination or tissue collection. The following tables summarize tests, methods, and abbreviations used in hematology tests (Table 3), coagulation tests (Table 4), clinical chemistry tests (Table 5), and urinalysis (Table 6).

TABLE 3
Advia 120, Hematology Analyzer

| TEST | METHOD | ABBREVIATIONS | UNITS |
|---|---|---|---|
| Red Blood | RBC are | RBC | ×10¹²/L |
| Cell Count | isovolumetrically | | |
| | sphered and | | |
| | measured by laser | | |
| | diode light source | | |
| Hemoglobin | Colorimetric/ | HGB | g/L |
| | Modified | | |
| | cyanmethemoglobin | | |
| Hematocrit | Calculated | HCT | L/L |
| Mean Corpuscular | Laser diode light | MCV | fL |
| Volume | source | | |
| Mean Corpuscular | Calculated | MCH | pg |
| Hemoglobin | | | |
| Mean Corpuscular | Calculated | MCHC | g/L |
| Hemoglobin | | | |
| Concentration | | | |
| Hemottlobin | Calculated | HDW | g/L |
| distribution width | | | |
| Platelet | Laser diode light | PLT | ×10⁹/L |
| | source | | |
| Mean platelet | Calculated | MPV | fL |
| volume | | | |
| Red Cell | Calculated | RDW | % |
| Distribution Width | | | |
| Reticulocyte Count | Stained using | RET | ×10¹²/L |
| Absolute and | Oxazine 750 and | | % |
| Relative | counted by laser | | |
| | diode light source | | |
| White Blood Cell | Basophil-laser | WBC | ×10⁹/L |
| Count | light source | | |
| | Peroxidase as | | |
| | secondary count | | |
| Neutrophil Count | Peroxidase | NEUT | ×10⁹/L |
| Absolute and | | | % |
| Relative | | | |
| Lymphocyte Count | Peroxidase | LYM | ×10⁹/L |
| Absolute and | | | % |
| Relative | | | |
| Monocyte Count | Peroxidase | MON | ×10⁹/L |
| Absolute and | | | % |
| Relative | | | |
| Eosinophil Count | Peroxidase | EOS | ×10⁹/L |
| Absolute and | | | % |
| Relative | | | |
| Basophil Count | Basophil-laser light | BAS | ×10⁹/L |
| Absolute and | source Peroxidase | | % |
| Relative | as secondary count | | |
| Large Unstained | Peroxidase | LUC | ×10⁹/L |
| Cells Absolute | | | % |
| and Relative | | | |

OSM3 Hemoximeter

| TEST | METHOD | ABBREVIATIONS | UNITS |
|---|---|---|---|
| Methemoalobin | Calculated | MetHb | % |

Blood Smear, Modified Wright's Stain

| TEST | ABBREVIATIONS | UNITS |
|---|---|---|
| Anisocytosis | ANISO | 1+ = slight (2-10 cells/ |
| | | HPF (high power field)) |
| Hypochromia | HYPO | 2+ = moderate (11-20 cells/ |
| | | HPF (high power field)) |
| Hyperchromia | HYPER | 3+ = severe (>20 cells/ |
| | | HPF (high power field)) |
| Macrocytosis | MACRO | PR = Presence (<2) |
| Microcytosis | MICRO | |
| Platelet Clump | PLT CLUMPS | |
| | reported as PLTC | |
| Howell-Jolly body | HJB | |
| Reactive | ATYPS reported | |
| Lymphocyte | as RL | |
| Polychromasia | HC VAR | |
| | reported as POLY | |
| Toxic Granulation | TOXG | |
| Ghost Cells | GHO | |
| Hypersegmented | HNEUT | |
| Neutrophil | | |
| Large Platelet | LARGE PLT | |
| | reported as LPLT | |
| Red Blood Cell | RBCF | |
| Fragment | | |
| Red Blood Cell | RBCP | Negative (N) or Positive (P) |
| Parasite | | |
| Nucleated Red | NRBC | Number of NRBC |
| Blood Cell | | in 100 WBC |

TABLE 4
ACL 100, Coagulation Analyzer

| TEST | METHOD | ABBREVIATIONS | UNITS |
|---|---|---|---|
| Protluombin Time | Coagulometric | PT | sec/Second |
| Activated Partial | Coagulometric | APTT | sec/Second |
| Thromboplastin | | | |
| Time | | | |
| Fibrinogen | Coagulometric | FIB | g/L |
| | (delta) | | |
| | measurement | | |

TABLE 5
Hitachi 912, Biochemistry Analyzer

| TEST | METHOD | SAMPLE | ABBREVIATIONS | UNITS |
|---|---|---|---|---|
| Albumin/Globulin | Calculated | | A/G | None |
| Albumin | Bromcresol green | Serum | ALB | g/L |
| Albumin -Rabbit | Bromcresol green | Serum | ALB | g/L |
| | (correction factor for rabbit) | | | |
| Alanine Aminotransferase | Coupled ALT/LDH | Serum | ALT | U/L |
| Alkaline Phosphatase | p-nitrophenylphosphate | Serum | ALP | U/L |
| Amylase | Blocked PNP-Maltoheptaoside method | Serum | AMY | U/L |
| Aspartate Aminotransferase | Coupled AST/MDH | Serum | AST | U/L |
| Bilirubin, Total | DPD (modified Jendrassik-Grof) | Serum | TBILI | μmol/L |
| Bilirubin. Direct | Azobilirubin | Serum | BIL-D | μmol/L |
| | (modified Jendrassik-Grof factored) | | | |
| Bilirubin, Indirect | Calculated | | BILI | μmol/L |
| Calcium. Total | O-Cresolphthalein complexone | Serum/Urine | CA | mmol/L |
| Chloride | Indirect ion selective electrode | Serum/Urine | CL | mmoI/L |
| Cholesterol, Total | Esterase/oxidase/peroxidase | Serum | CHOL | mmol/L |
| Creatinine | Alkaline picrate kinetic, compensated | Serum/Urine | CRE | μmol/L |
| Creatinine Clearance | Calculated | | CRE CL | ml/min/k |
| Creatine kinase | Coupled hexokinase/G6P-DH kinetic | Serum | CK | U/L |
| Gamma-glutamyltransferase | α-glutamyl-p-nitro-anilide | Serum | GGT | U/L |
| Globulin | Calculated | | GLOB | g/L |
| Glucose | Hexokinase | Serum/Urine | GUI | mmol/L |
| Lactate Dehydrogenase | Lactate oxidase/peroxidase | Serum | LDH | U/L |
| Phosphorus Inorganic | Phosphomolybdate complex | Serum/Urine | PHOS | mmol/L |
| Potassium | Indirect ion selective electrode | Serum/Urine | K | mmol/L |
| Sodium | Indirect ion selective electrode | Serum/Urine | NA | mmol/L |
| Total Protein | Biuret | Serum | TP | g/L |
| | Benzethonium chloride | Urine | TP | |
| Triglycerides | GK/GPO/POD | Serum | TRIG | mmol/L |
| Urea | Urease UV/GLDH kinetic | Serum/Urine | UREA | mmol/L |
| Urea Nitrogen | Urease UV/GLDH kinetic/.357 | Serum | UREAN | mg/dL |
| HDL Cholesterol | Enzymatic/Colorimetric | Serum | HDL | mmol/L |
| | (Esterase/Oxidase coupled with PEG/Peroxidase) | | | |
| LDL Cholesterol | Enzymatic/Colorimetric | Serum | LDL | mmol/L |
| | (Esterase/Oxidase/Peroxidase) | | | |
| Sorbitol Dehydroeenase | Oxidation Reduction between sorbitol | Serum | SDH | U/L |
| | and fructose | | | |
| Magnesium | Enzymatic | Serum | MG | mmol/L |
| | | Urine | | mmol/L |
| Carbon dioxide/bicarbonate | Phosphenolpynwate carboxylase | Serum | PEPC | mmol/L |
| | CO2/HCO3 | | | |

TABLE 6

| TEST | METHOD | ABBREVIATIONS | UNITS | PRINTED RESULTS | |
|---|---|---|---|---|---|
| Glucose | Glucose-Oxidase/ | GLU | mmol/L | Negative | neg |
| | Peroxidase | | | 5.5 | |
| | | | | 14 | |
| | | | | 28 | |
| | | | | ≧55 | |
| Bilirubin | Coupled bilirubin/ | BIL | | Negative | neg |
| | diazotized | | | Small | small |
| | dichloroaniline | | | Moderate | MOD |
| | | | | Large | LAR |
| Ketone | Reaction between | KET | mmol/L | Negative | neg |
| | acetoacetic/nitroprusside | | | Trace | trace |
| | | | | 1.5 | |
| | | | | 3.9 | |
| | | | | ≧7.8 | |
| Blood | Peroxidase activity | BLD | Ery/μL | Negative | neg |
| | of hemoglobin which | | | Trace-lyzed | TR-LY |
| | catalyzes the reaction of | | | Trace-Intact | TR-IN |
| | cumene hydroperoxide | | | Ca = circa | Ca 25 |
| | and 3.3′, 5.5′- | | | | Ca 80 |
| | tetramethylbenzidine | | | | Ca 200 |
| pH | Double indicator activity | PH | | 5.0 | 7.0 |
| | | | | 5.5 | 7.5 |
| | | | | 6.0 | 8.0 |
| | | | | 6.5 | 8.5 |
| | | | | | ≧9.0 |
| Protein | Protein-error-of- | Prot | g/L | Negative | neg |
| | indicators principle | | | Trace | trace |
| | | | | 0.3 | |
| | | | | 1.0 | |
| | | | | ≧3.0 | |
| Urobilinogen | Modified Ehrlich | UBG | μmol/L | 3.2 | |
| | | | | 16 | |
| | | | | 33 | |
| | | | | 66 | |
| | | | | ≧131 | |
| Nitrite | Conversion of nitrate | NIT | | Negative | N |
| | to nitrite by the action | | | Positive | P |
| | of Grain negative | | | | |
| | bacteria in urine | | | | |
| Leucocyte | Hydrolization of the | LEU | Leu/μL | Negative | N |
| | derivatized pyrrole | | | Ca = circa | Ca 15 |
| | amino ester acid then | | | | Ca 70 |
| | reacts with a | | | | Ca 125 |
| | diazonitun salt | | | | Ca 500 |

Atago Refractometer

| TEST | METHOD | ABBREVIATIONS | PRINTED RESULTS |
|---|---|---|---|
| Specific Gravity | Refractometer | SG | 1.000 to 1.098 |

Wescor, Osmometer

| TEST | METHOD | ABBREVIATIONS | UNITS |
|---|---|---|---|
| Osmolality | Vapor Pressure | OSMO-U | mmol/kg |

Macroscopic Analysis

| TEST | METHOD | REPORTED RESULTS | ABBREVIATIONS |
|---|---|---|---|
| Color (UCOL) | Reflectance | Yellow | yell |
| | | Orange | Or |
| | | Red | R |
| | | Green | Gr |
| | | Blue | Bl |
| | | Brown | Br |
| | | Light | It |
| | | Dark | dk |
| Appearance (CLA) | | Clear | C |
| | | Cloudy | Cl |
| | | Turbid | T |

Microscopic Analysis

| | | |
|---|---|---|
| TEST | ABBREVIATIONS | REPORTED RESULTS |

ORGANIZED SEDIMENTS

| | | |
|---|---|---|
| Cells | | |
| White Blood Cell | WBC | (-) or in number/HPF(high power field) |
| Red Blood Cell | RBC | (-) or in number/HPF(high power field) |
| Epithelial Cell | EPIT | (-). 1+, 2+, 3+ |
| Fat Bodies | FATB | (-). 1+, 2+. 3+ |
| Pus | | (-). 1+, 2+, 3+ |
| Other | | |
| Casts | | (-) or in number/LPF(low power field) |
| Granular | G | |
| Hyaline | HY | |
| RBC | | |
| WBC | | |
| Waxy | WXY | |
| Miscellaneous | | |
| Trichomonas | TRIC | (-), PR. 1+. 2+, 3+ |
| Bacteria | UBAC or BCT | (-). 1+, 2+, 3+ |
| Mucous | MUCO | (-), 1+, 2+, 3+ |
| Sperm | SPER or SPM | (-). 1+, 2+, 3+ |
| Yeast | YST | (-), PR, 1+, 2+, 3+ |

UNORGANIZED SEDIMENTS

| | | |
|---|---|---|
| Crystals | | (-), 1+, 2+, 3+ |
| Ammonium urate | AmU | |
| Amorphous phosphate | AP | |
| Amorphous urate | AU | |
| Bilinthin crystal | BC | |
| Calcium carbonate | CC | |
| Calcium oxalate | CO | |
| Calcium phosphate | CP | |
| Calcium sulfate | CS | |
| Hippuric acid | HA | |
| Leucine crystal | LC | |
| Triple phosphate | TP | |
| Tyrosine crystal | TC | |
| Unidentified crustals | UC | |
| Uric acid | UA | |

### Results

### Analysis of Dosing Formulations

Analyses determined that all formulations were within acceptance criteria ranges (±10%). The formulation concentrations ranged between 94.4 and 106.4% of nominal concentrations and therefore considered acceptable. Although no formal formulation stability under conditions of use or at 4° C. are available, results from formulations stored refrigerated for at least 48 hours prior to analysis or at room temperature on the day of preparation, demonstrated all formulation concentrations were stable.

### Test Atmosphere Concentration and Estimated Achieved Dose Levels

Table 7 shows achieved test atmosphere concentrations.

TABLE 7

| | | Projected | Achieved Mean | | |
|---|---|---|---|---|---|
| | | Riboflavin 5′- | Riboflavin 5′- | | |
| | | Phosphate Aerosol | Phosphate Aerosol | | |
| Group | Group | Concentration | Concentration | | % of Target |
| Number | Designation | (mg/L) | (mg/L) | % CV | Concentration |
| 1 | Vehicle control | 0.000 | 0 | NA | NA |
| 2 | Low Dose | 0.006 | 0.0064 | 7.3 | 106.0 |
| 3 | Mid Dose | 0.024 | 0.0254 | 8.8 | 105.9 |
| 4 | High Dose | 0.060 | 0.0655 | 9.0 | 109.1 |

NA = Not applicable

The overall achieved aerosol concentrations for Groups 2 to 4 were within 10% of the targeted Riboflavin 5′-Phosphate concentrations. The generated atmosphere for all Riboflavin 5′-Phosphate groups was stable over 28 days with % CV between 7.3 and 9.0%.

TABLE 8

| | Group 2 | Group 3 | Group 4 |
|---|---|---|---|
| Levels of | Low Dose | Mid Dose | High Dose |
| Exposure | Achieved Mean Riboflavin 5′- | | |
| Chamber | Phosphate Aerosol Concentration (mg/L) | | |
| Top | 0.0056 | 0.0235 | 0.0556 |
| Middle | 0.0057 | 0.0227 | 0.0569 |
| Bottom | 0.0056 | 0.0216 | 0.0550 |
| Mean | 0.0056 | 0.0226 | 0.0558 |
| SD | 0.00006 | 0.00095 | 0.00097 |
| CV (%) | 1.1 | 4.2 | 1.7 |

The aerosols obtained from the top, middle and bottom tiers from Groups 2 to 4 were considered homogeneous.

TABLE 9

| | | Projected | | | | Mean | Mean |
|---|---|---|---|---|---|---|---|
| | | dose level | Duration of | | Mean body | estimated | achieved |
| Group | Group | of Riboflavin | exposure | | weight | achieved dose | respirable dose |
| Number | Designation | 5′-phosphate | (min) | Sex | (kg)^{a} | (mg/kg/day)^{b} | (mg/kg/day)^{c} |
| 1 | Vehicle | 0 | 60 | Male | 0.342 | 0 | 0 |
| | control | | | Female | 0.244 | 0 | |
| | | | | Combined | 0.293 | 0 | |
| 2 | Low dose | 0.24 | 60 | Male | 0.330 | 0.237 | 0.024 |
| | | | | Female | 0.251 | 0.250 | |
| | | | | Combined | 0.291 | 0.244 | |
| 3 | Mid dose | 0.96 | 60 | Male | 0.371 | 0.947 | 0.097 |
| | | | | Female | 0.249 | 0.992 | |
| | | | | Combined | 0.280 | 0.969 | |
| 4 | High dose | 2.40 | 60 | Male | 0.346 | 2.402 | 0.248 |
| | | | | Female | 0.250 | 2.556 | |
| | | | | Combined | 0.298 | 2.479 | |

^{a}Calculated using the mean body weights obtained from Days 1 to 28.
^{b}Calculated using mean achieved aerosol concentrations from Days 1 to 28.
^{c}Calculated based on FDA assumed deposition fraction of 25% of mean estimated achieved doses within the respiratory tract.

The overall estimated achieved doses for all groups were acceptable and within 3.3% of the projected dose levels. For TK evaluation, the mean body weights and the achieved Riboflavin 5′-Phosphate aerosol concentrations from each respective TK day were used to calculate the achieved dose levels. Table 10 shows achieved Dose levels of Riboflavin 5′-Phosphate on Day 1.

TABLE 10

| | | Projected | | | | Mean | Mean |
|---|---|---|---|---|---|---|---|
| | | dose level | Duration of | | Mean body | estimated | achieved |
| Group | Group | of Riboflavin | exposure | | weight | achieved dose | respirable dose |
| Number | Designation | 5′-phosphate | (min) | Sex | (kg)^{a} | (mg/kg/day)^{b} | (mg/kg/day)^{c} |
| 1 | Vehicle | 0 | 60 | Male | 0.247 | 0 | 0 |
| | control | | | Female | 0.196 | 0 | |
| | | | | Combined | 0.222 | 0 | |
| 2 | Low dose | 0.24 | 60 | Male | 0.242 | 0.283 | 0.029 |
| | | | | Female | 0.203 | 0.292 | |
| | | | | Combined | 0.223 | 0.288 | |
| 3 | Mid dose | 0.96 | 60 | Male | 0.236 | 1.041 | 0.105 |
| | | | | Female | 0.212 | 1.063 | |
| | | | | Combined | 0.224 | 1.052 | |
| 4 | High dose | 2.40 | 60 | Male | 0.276 | 2.592 | 0.266 |
| | | | | Female | 0.213 | 2.723 | |
| | | | | Combined | 0.245 | 2.658 | |

^{a}Calculated using the mean body weights obtained from Day −1.
^{b}Calculated using mean achieved aerosol concentrations from Day 1.
^{c}Calculated based on FDA assumed deposition fraction of 10% of mean estimated achieved doses within the respiratory tract.

Table 11 shows Achieved Dose levels of Riboflavin 5′-Phosphate on Day 28.

TABLE 11

| | | Projected | | | | Mean | Mean |
|---|---|---|---|---|---|---|---|
| | | dose level | Duration of | | Mean body | estimated | achieved |
| Group | Group | of Riboflavin | exposure | | weight | achieved dose | respirable dose |
| Number | Designation | 5′-phosphate | (min) | Sex | (kg)^{a} | (mg/kg/day)^{b} | (mg/kg/day)^{c} |
| 1 | Vehicle | 0 | 60 | Male | 0.414 | 0 | 0 |
| | control | | | Female | 0.273 | 0 | |
| | | | | Combined | 0.344 | 0 | |
| 2 | Low dose | 0.24 | 60 | Male | 0.401 | 0.283 | 0.024 |
| | | | | Female | 0.286 | 0.292 | |
| | | | | Combined | 0. | 0.288 | |
| 3 | Mid dose | 0.96 | 60 | Male | 0.411 | 1.041 | 0.100 |
| | | | | Female | 0.281 | 1.063 | |
| | | | | Combined | 0.224 | 1.052 | |
| 4 | High dose | 2.40 | 60 | Male | 0.276 | 2.592 | 0.233 |
| | | | | Female | 0.213 | 2.723 | |
| | | | | Combined | 0.245 | 2.658 | |

^{a}Calculated using the mean body weights obtained from Day 28.
^{b}Calculated using mean achieved aerosol concentrations from Day 28.
^{c}Calculated based on FDA assumed deposition fraction of 10% of mean estimated achieved doses within the respiratory tract.

### Particle Size Distribution

Particle size distribution measurements are summarized in Table 12.

TABLE 12

| | | Riboflavin 5′Phosphate | | |
|---|---|---|---|---|
| Group | Group | Mean Particle Size Data | | |
| Number | Designation | MMAD | σg | % of particles <3.1 μm |
| 2 | Low Dose | 1.2 | 2.40 | 91.8 |
| 3 | Mid Dose | 1.2 | 2.27 | 91.9 |
| 4 | High Dose | 1.2 | 2.26 | 87.8 |

* For Group 1 (Vehicle Control), a Mean MMAD of 1.7 μm with a σg of 2.33 μm was obtained by gravimetric determination
MMAD = Mass median aerodynamic diameter (μm)
σg = Geometric standard deviation.

Particle size distribution measurements confirmed that the aerosolized formulations of Riboflavin 5′-Phosphate were respirable for the rat, and the deposition within the respiratory tract was considered to be 100%. The corresponding gravimetrically determined control aerosol was considered respirable and comparable to those of the treated Riboflavin 5′-Phosphate groups.

### Toxicokinetics

Riboflavin 5′-phosphate concentrations were detected in only very few treated animals following single or repeated exposures to riboflavin 5′-phosphate, with measured concentrations generally only slightly above the quantifiable limit and no kinetic parameters were calculated.

### Day 1

There were no measurable concentrations of riboflavin 5′-phosphate or its metabolite (riboflavin) in the control group (Group 1) on Day 1. There were also no measurable concentrations of riboflavin 5′-phosphate or riboflavin in the low dose group on Day 1 except for one female rat at the 24 hour mark which had a riboflavin concentration of 0.829 μg/mL. There was a slight increase in plasma concentration of riboflavin from the mid to high dose groups in both sexes. The Cₘₐₓ values were 0.0782 and 0.146 μg/mL for males and 0.0491 and 0.124 μg/mL for females for mid and high dose groups, respectively. The corresponding AUC_{(0-T)} values were 0.0159 and 0.104 μg·h/mL for males and 0.0096 and 0.0842 μg·h/mL for females, respectively. The concentration values were slightly higher in males than in females for Day 1. The male to female Cₘₐₓ and AUC_{(0-T)} ratios were 1.59 and 1.18 for the mid dose and 1.65 and 1.24 for the high dose group.

### Day 28

There were no measurable concentrations of riboflavin 5′-phosphate or its metabolite (riboflavin) in the control group (Group 1) on Day 28. There were also no measurable concentrations of riboflavin 5′-phosphate or riboflavin in the low dose group. The Cₘₐₓ values were 0.266 and 0.117 μg/mL for males and 0.0735 and 0.145 μg/mL for females for the mid and high dose groups, respectively. The AUC_{(0-T)} values were 0.117 μg·h/mL for high dose males and 0.0121 and 0.0566 μg·h/mL for females of the mid and high dose groups, respectively. Overall, the plasma concentrations of riboflavin (Cₘₐₓ and AUC_{(0-T)}) did not increase as a function of the administered doses of riboflavin 5′-phosphate in both sexes. Similarly, no accumulation of riboflavin was observed following 28 daily repeated dosing in both sexes. Day 28 to Day 1 Cₘₐₓ and AUC_{(0-T)} ratios of riboflavin in males were 0.801 and 0.433 for the high dose group, respectively. Day 28 to Day 1 Cmax and AUC_{(0-T)} ratios of riboflavin in females were 1.50 and 1.17 and 1.25 and 0.672 for the mid and high dose groups, respectively.

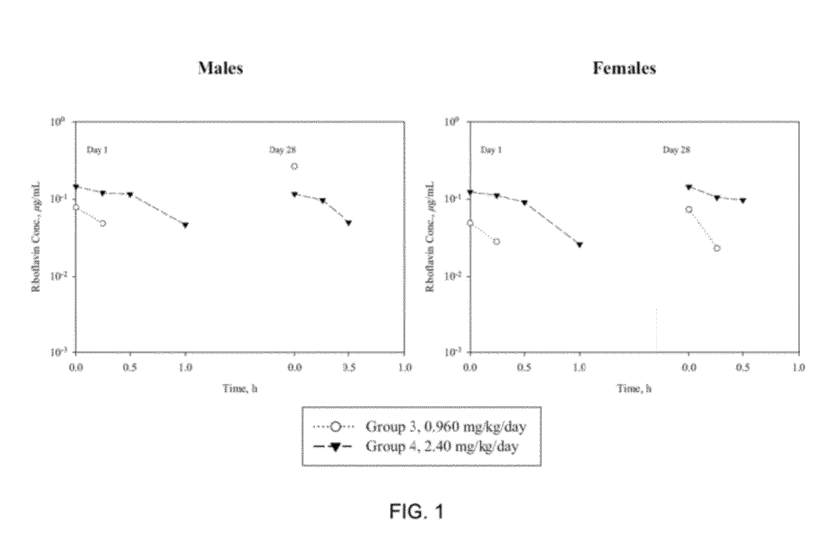

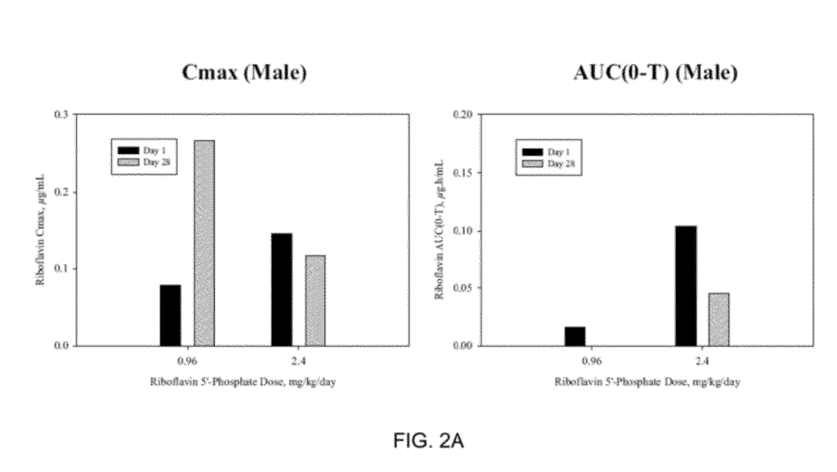

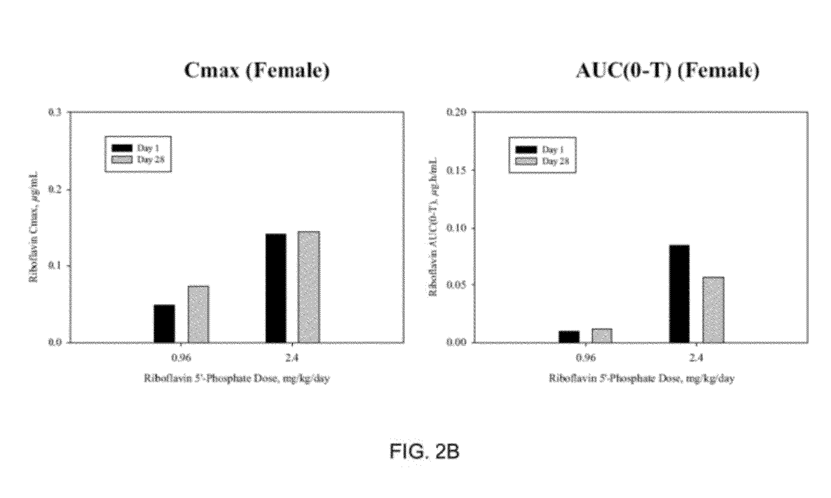

FIG. 1 shows graphs for mean riboflavin plasma concentration-time profiles in male or female rats following aerosolized riboflavin 5′-phosphate for 28 days. FIG. 2A shows graphs for mean riboflavin Cₘₐₓ and AUC_{(0-T)} in male rats following aerosolized doses of riboflavin 5′-phosphate for 28 days. FIG. 2B shows graphs for mean riboflavin Cₘₐₓ and AUC_{(0-T)} in female rats following aerosolized doses of riboflavin 5′-phosphate for 28 days. Table 13 summarizes toxicokinetic parameters of riboflavin in rats following aerosolized doses of riboflavin 5′-phosphate for 28 days.

TABLE 13

| | Riboflavin | Male | | | Female | | |
|---|---|---|---|---|---|---|---|
| Day | 5′-phosphate | 0.24 | 0.96 | 2.4 | 0.24 | 0.96 | 2.4 |
| 1 | Cₘₐₓ (μg/ml) | N/A | 0.0782 | 0.146 | N/A | 0.0491 | 0.124 |
| | Tmax (h)^{a} | N/A | 0.00 | 0.00 | N/A | 0.00 | 0.00 |
| | AUC(0-T) | N/A | 0.0159 | 0.104 | N/A | 0.00966 | 0.0842 |
| | (μg · h/ml) | | | | | | |
| | Cmax ratio | N/A | 1.59 | 1.18 | — | — | — |
| | (M/F)^{b} | | | | | | |
| | AUC ratio | N/A | 1.65 | 1.24 | — | — | — |
| | (M/F)^{c} | | | | | | |
| 28 | Cₘₐₓ (μg/ml) | N/A | 0.266 | 0.117 | N/A | 0.0735 | 0.145 |
| | Tmax (h)^{a} | N/A | 0.00 | 0.00 | N/A | 0.00 | 0.00 |
| | AUC(0-T) | N/A | N/A | 0.0450 | N/A | 0.0121 | 0.0566 |
| | (μg · h/ml) | | | | | | |
| | Cmax ratio | N/A | N/A | 0.801 | N/A | 1.50 | 1.17 |
| | (M/F)^{d} | | | | | | |
| | AUC ratio | N/A | N/A | 0.433 | N/A | 1.25 | 0.672 |
| | (M/F)^{e} | | | | | | |

^{a}Cₘₐₓmale/Cₘₐₓfemale
^{b}AUC_{(0-T)}male/AUC_{(0-T)}female
^{c}CₘₐₓDay 28/CₘₐₓDay 1
^{d}AUC_{(0-T)}Day 28/AUC_{(0-T)}Day 1
N/A: Not Applicable.

### Functional Observation Battery

There were no changes noted in any functional observation battery parameters that were considered to be treatment-related for any animals during the study. Statistically significant differences (p≦0.05) from the control group were noted in some parameters. However, the differences from the vehicle control group were inconsistent between sexes and showed no relationship to dose level and were considered incidental and unrelated to treatment.

### Clinical Pathology

### Hematology

There were no changes noted in any haematology parameters that were considered to be treatment-related for any animals during the study. Statistically significant differences (p≦0.05) from the control group were noted in a number of parameters. However, the differences from the vehicle control group were inconsistent between sexes and showed no relationship to dose level and were considered incidental and unrelated to treatment.

### Coagulation

Coagulation times were unaffected by treatment.

### Clinical Chemistry

There were no changes noted in any clinical chemistry parameters that were considered to be treatment-related for any animals during the study. Statistically significant differences (p≦0.05) from the control group were noted in a number of parameters. However, the differences from the vehicle control group were inconsistent between sexes and showed no relationship to dose level and were considered incidental and unrelated to treatment.

### Urinalysis

There were no changes in any of the urinalysis parameters evaluated that were considered affected by treatment.

### Organ Weights

There were no changes noted in any organ weights (absolute or relative to body weight) that were considered to be treatment-related for any animals during the study. Statistically significant differences (p≦0.05) from the control group were noted in the heart (relative) and prostate (absolute and relative) of Groups 2 and 3 male animals. In addition, other statistically significant differences (p≦0.05) from the control group were noted in a number of organs. However, the differences from the vehicle control group were inconsistent between sexes and showed no relationship to dose level and were considered incidental and unrelated to treatment.

### Macroscopic Findings

There were no findings attributed to the test article administration and all findings observed at necropsy were considered to be incidental in origin and of no biological significance.

### Microscopic Findings

There were no microscopic findings that were considered to be test article related. All changes were not considered toxicologically significant as they were agonal, not dose-related, of low incidence or severity, occurred in control and treated animals, or are incidental in this age and strain of laboratory rats.

### Discussion

The primary purpose of this study was to determine the toxicity and toxicokinetic profile of the test article, riboflavin 5′-phosphate, following inhalation (nose-only) administration to rats for 28 consecutive days. Riboflavin 5′-phosphate was well tolerated and there were no adverse clinical observations detected by assessment of functional observation battery and clinical pathology parameters or following histopathological examination of all major organs. The lymphoid tissues, including spleen, thymus, lymph node mandibular and lymph node mesenteric, and respiratory tract tissues including nasal cavity, nasopharynx, larynx, lymph node bronchial, carina, trachea, lungs and bronchi, were examined histologically and there were no indications of local or systemic toxicity.

One male rat dosed at 0.244 mg/kg/day was found dead on Day 12. A cause of death was considered accidental and related to restraint procedures and was not considered to be test article related as mortality was limited to a single low dose rat. Due to the lack of measurable plasma concentrations of riboflavin 5′-phosphate in treated animals no kinetics were calculated. Concentrations of the metabolite (riboflavin) were measurable in the plasma of both sexes and exposures (Cₘₐₓ and AUC_{(0-T)}) appeared to be dose proportional. Overall, there was no gender difference in riboflavin exposure and there was no accumulation following repeated dosing of riboflavin 5′-phosphate for 28 days.

In conclusion, the No Observed Effect Level (NOEL) in Sprague-Dawley rats after once daily inhaled administration, of the test article, riboflavin 5′-phosphate, is considered to be at the high dose group tested at 2.479 mg/kg/day for 28 consecutive days.

### Example 2

### Riboflavin 5′-phosphate: A 28-Day Aerosolized Liquid Inhalation Toxicity Study in Beagle Dogs

The objective of the study was to determine the toxicity and toxicokinetic profile of the test article, Riboflavin 5′-phosphate, following inhalation (oronasal) administration to dogs for 28 consecutive days.

### Experimental Design

TABLE 14

| | | Projected Dose | Projected | | |
|---|---|---|---|---|---|
| | | Level of | Respirable | | |
| | | Riboflavin | Delivered | Number of | |
| Group | Group | 5′-phosphate ^{a} | Dose ^{b} | Animals | |
| Number | Designation | (mg/kg/day) | (mg/kg/day) | Male | Female |
| 1 | Vehicle | 0 | 0 | 3 | 3 |
| | Control | | | | |
| 2 | Low Dose | 0.12 | 0.03 | 3 | 3 |
| 3 | Mid Dose | 0.48 | 0.12 | 3 | 3 |
| 4 | High Dose | 1.20 | 0.3 | 3 | 3 |

^{a}: Projected dose levels are calculated based on an estimated body weight of 10.0 kg.
^{b}: FDA assumed deposition of 25%.

Test article: riboflavin 5′-phosphate sodium salt hydrate; alternate identity: vitamin B₂ phosphate; description: yellow crystals; potency: 74%. Vehicle article: saline (0.9% (w/v) NaCl for injection); description: clear colorless solution. Exposure method: inhalation by oronasal face mask exposure.

### Preparation of Test and Control/Vehicle Articles Formulations

The test (0.5 mg/mL, 2.0 mg/mL, 5.0 mg/mL) and/or vehicle control formulations (0 mg/mL) for each group were prepared fresh daily on each day by dissolving the test article in saline. All prepared test article formulations were protected from light and kept at room temperature (RT).

### Treatment

Duration of treatment: 28 days. Test system: Dog (Canis familiaris); strain: Beagle. Projected aerosol concentrations and dose levels are summarized in Table 15.

TABLE 15

| | | | | Projected | Projected | |
|---|---|---|---|---|---|---|
| | | Projected | Projected | Aerosol | Formulation | |
| | | Dose Level | Respirable | Concentration | Concentration | |
| | | of Riboflavin | Delivered | of Riboflavin | of Riboflavin | Duration of |
| Group | Group | 5′-phosphate ^{a} | Dose ^{b} | 5′-phosphate | 5′-phosphate | Exposure |
| Number | Designation | (mg/kg/day) | (mg/kg/day) | (mg/L) ^{c} | (mg/ml) | (min) |
| 1 | Vehicle | 0 | 0 | 0 | 0 | 60 |
| | Control | | | | | |
| 2 | Low Dose | 0.12 | 0.03 | 0.006 | 0.5 | 60 |
| 3 | Mid Dose | 0.48 | 0.12 | 0.024 | 2.0 | 60 |
| 4 | High Dose | 1.20 | 0.3 | 0.060 | 5.0 | 60 |

^{a} Projected dose levels were calculated based on an estimated body weight of 10.0 kg.
^{b} FDA assumed deposition of 25%
^{c} Projected aerosol concentrations were determined based on the results obtained from a technical validation study.

### Estimation of Achieved Dose Levels

Achieved dose levels during the exposure period were estimated using the methods described in Example 1.

### Inhalation System

The aerosol was produced by metering the flow of the test and control article formulations to 3 clinical nebulizers (Sidestream). The aerosol produced was discharged through a 40 mm diameter tube into a flow-past inhalation exposure system. The airflow rate through the exposure system was monitored and recorded manually during the aerosol generation. Airflow to the exposure system was controlled by the absolute volume of air supplying the aerosol generators using variable area flow meters. Control of the aerosol exhaust flow from the animal exposure system was achieved using an exhaust valve. The system provided a minimum of 6 L/min of aerosol to each animal exposure position and the inlet and outlet airflows were balanced to ensure that there was no dilution of the generated aerosol by air drawn from the environment. Any minor variations in flow were buffered by a balloon reservoir. An equal delivery of aerosol to each exposure position was achieved by employing a distribution network that was identical for each individual exposure position attached to the system.

### Inhalation System Monitoring

Determinations of aerosol concentration, particle size distribution, oxygen concentration, relative humidity and temperature were performed on test atmosphere samples collected from a representative port of the exposure system, with a collection sample flow-rate of 1 L/min. The sample flow rates were precisely controlled using variable area flow meters that were calibrated before use using a primary airflow calibrator. The absolute volume of each aerosol concentration sample was measured using a wet type gas meter.

### Determination of Aerosol Concentration

During the treatment period, multiple aerosol concentration samples were collected on a daily basis onto filters from all groups, including the control group, for each aerosol generation occasion.

### Particle Size Distribution and Mass Median Aerodynamic Diameter (MMAD)

The distribution of particle size in the generated aerosols for Groups 2 to 4 were measured weekly during the treatment period by collecting samples into a 7-Stage Mercer Cascade Impactor and the particle size of aerosolized Riboflavin 5′-phosphate was determined using a validated analytical. The distribution of particle size in the generated aerosols for Group 1 were estimated once weekly by gravimetric determination. The MMAD and the Geometric Standard Deviation (GSD) were calculated based on the results obtained from the impactor using a log-probit transformation.

### In-Life Observations

Only the data collected during the 1 week period immediately prior to treatment were reported for the pre-treatment period.

### Mortality

Mortality checks were performed once daily during the acclimation and pre-treatment periods, and twice a day (am and pm) during the treatment period of the study.

### Clinical Observations

Cage-side clinical signs (ill health, behavioral changes etc.) were recorded for all animals once daily during the acclimation period and twice daily (pre-exposure and post-exposure) during the treatment period except on detailed clinical examination days. A detailed clinical examination of each dog was performed (replacing a cage-side clinical sign observation in the morning) at least once pretreatment, one day prior to Day 1, weekly during the treatment period and before necropsy.

### Body Weights

Body weights were recorded for all animals at least once prior to group assignment, and approximately one week prior to initiation of treatment. Body weights were recorded for all animals one day prior to Day 1 and weekly during the treatment period, as well as terminally.

### Food Consumption

Individual daily food intake was recorded for all animals during the last week of the pretreatment period and throughout the treatment period.

### Ophthalmoscopy

Funduscopic (indirect ophthalmoscopy) and biomicroscopic (slit lamp) examinations were performed for all animals, once during the pre-treatment period and during Week 4 of the treatment period.

### Electrocardiography (ECG) and Blood Pressure

Electrocardiograms (limb leads I, II and III, and augmented leads aVR, aVL and aVF) were obtained for all dogs once during the pre-treatment period and at least 90 minutes (between 90-120 minutes) postexposure on Days 1 and during Week 4 of the treatment period. In addition, indirect blood pressure was measured on the same occasions as ECG using a tail cuff. The tracings were assessed for gross changes indicative of cardiac electrical dysfunction and the potential presence of abnormalities involving heart rate (lead II), sinus and atrioventricular rhythm or conductivity were determined. Heart rate, PR interval, QRS duration, QT and QTc intervals values were tabulated for incorporation into the study report. A sling was utilized to restrain each animal during the recording of its ECG. ECGs' were evaluated by a consultant in veterinary cardiology.

### Respiratory Parameter Measurements

Respiratory parameters (tidal volume, respiration rate and minute volume) were obtained from all animals once during the pre-treatment period for at least 15 minutes, and for up to 90 minutes (an average of 15 minute interval was recorded) from the end of exposure during Week 4 of the treatment period. For some animals, the data acquisition was started within 35 minutes post end of exposure instead of the targeted 15 minutes from the end of exposure due to animal vocalization/excessive animal movement which occurred during the hardware calibration. Such disturbances during respiratory measurements are common and unavoidable and the deviation has no impact on the integrity of the study design or the overall interpretation of the data. The data was acquired using the NOTOCORD/LifeShirt Wireless system. Prior to data collection, the dogs were acclimated to the LifeShirt and protective jacket for 3 days for increasing periods up to 90 minutes.

### Toxicokinetics

A series of 9 blood samples (approximately 2.0 mL each) were removed from each dog on each of Days 1 and 28 of the treatment period. For this purpose, each dog was bled by venipuncture and the samples were collected into tubes containing the anticoagulant, Sodium Heparin. Tubes were placed immediately on wet ice pending processing within 30 minutes of collection. On each occasion, samples were collected at pre-dose, immediately post exposure (IPD), 15 and 30 minutes, 1, 2, 4, 6 and 24 hours post exposure. Following collection, the samples were centrifuged (approximately 4° C.) and the resulting plasma was recovered and stored frozen at ITR (approximately −20° C.) in labeled tubes protected from light pending shipment (on dry ice).

### Clinical Pathology

### Blood/Urine Sampling

Laboratory investigations (hematology, coagulation, clinical chemistry and urinalysis) were performed on all animals prior to the start of treatment and at the end of treatment (Day 29). Blood samples were collected by venipuncture following an overnight period of food deprivation consisting of at least 12 hours. Urine was collected from animals deprived of food and water, overnight (at least 16 hours, but for water, no more than 20 hours).

### Hematology

The following parameters were measured on blood samples (nominal 1 mL) collected into EDTA anticoagulant:

| Red blood cell count | Platelet count |
|---|---|
| Mean Corpuscular | WBC differential |
| Hemoglobin (calculated) | (absolute) |
| Hematocrit (calculated) | Reticulocyte (absolute and percentage) |
| Mean Corpuscular Volume | Mean Corpuscular Hemoglobin |
| Hemoglobin | Concentration (calculated) |
| Morphology of cells | Heinz Body (stained blood smear)* |
| White blood cell count | |

*The blood smear slides were retained for possible future analysis but were subsequently not required for evaluation and were retained with the study data.

### Coagulation

The following parameters were measured on blood samples (nominal 1.3 mL) collected into citrate anticoagulant:
Activated partial thromboplastin time
Prothrombin time

### Clinical Chemistry

The following parameters were measured on blood samples (nominal 1.1 mL) collected into tubes containing a clotting activator:

| | |
|---|---|
| A/G ratio (calculated) | Aspartate aminotransferase |
| Globulin (calculated) | Potassium |
| Alanine aminotransferase | Bilirubin (total, direct and indirect) |
| Glucose | Sodium |
| Albumin | Calcium |
| Lactate Dehydrogenase | Total protein |
| Alkaline phosphatase | Chloride |
| Magnesium | Triglycerides |
| Amylase | Cholesterol (total) |
| Phosphorus (inorganic) | Urea |
| Creatinine | |

### Urinalysis

The following parameters were measured on urine samples:

| | |
|---|---|
| Bilirubin | Glucose |
| Protein | Urobilinogen |
| Blood | Ketones |
| Sediment microscopy | Volume |
| Color and appearance | pH |
| Specific gravity | |

### Data Evaluation and Statistics

Numerical data obtained during the conduct of the study were subjected to calculation of group means and standard deviations and were reported along with all individual numerical and non numerical results. The data (excluding ECG's) was analyzed for homogeneity of variance using Levene Median and for normality using Kolmogorov-Smirnov tests. Homogeneous data was analyzed using the Analysis of Variance and the significance of intergroup differences were analyzed using Dunnett's test. Heterogeneous data was analyzed using Kruskal-Wallis test and the significance of intergroup differences between the controls and treated groups were assessed using Dunn's test. A significance level of p≦0.05 was reported. The statistical analyses were performed compared to control, i.e. Groups 2 to 4 compared to Group 1. The numerical data was subjected to calculation of group means and standard deviations.

### Results

### Analysis of Dosing Formulations

Analyses determined that all formulations were within acceptance criteria ranges (±10%). The formulation concentrations ranged between 94.9 and 104.5% of nominal concentrations and were therefore considered acceptable. Although no formal formulation stability under conditions of use or at 4° C. are available, results from formulations stored refrigerated for at least 48 hours prior to analysis or at room temperature on the day of preparation, demonstrated all formulation concentrations were stable. Table 16 summarizes test atmosphere concentration and estimated achieved dose levels

TABLE 16

| | | Projected Riboflavin | | | |
|---|---|---|---|---|---|
| | Group | 5′-Phosphate | Achieved Mean | | |
| | Designation | Aerosol | Riboflavin | | |
| Group | Concentration | Concentration | 5′-Phosphate | | % of Target |
| Number | (mg/L) | (mg/L) | Aerosol | % CV | Concentration |
| 1 | Vehicle control | 0.000 | 0 | NA | NA |
| 2 | Low Dose | 0.006 | 0.0058 | 8.1 | 96.9 |
| 3 | Mid Dose | 0.024 | 0.0244 | 10.7 | 101.7 |
| 4 | High Dose | 0.060 | 0.0640 | 8.5 | 106.8 |

NA: Not applicable.

The overall achieved aerosol concentrations for Groups 2 to 4 were within 6.7% of the targeted riboflavin 5′-Phosphate concentrations. The generated atmosphere for all riboflavin 5′-phosphate groups was stable over 28 days with % CV between 8.1 and 10.7%. Table 17 summarizes overall estimated achieved dose levels in main study animals.

TABLE 17

| | | Projected | | | | Mean | Mean |
|---|---|---|---|---|---|---|---|
| | | dose level | Duration of | | Mean body | estimated | achieved |
| Group | Group | of Riboflavin | exposure | | weight | achieved dose | respirable dose |
| Number | Designation | 5′-phosphate | (min) | Sex | (kg)^{a} | (mg/kg/day)^{b} | (mg/kg/day)^{c} |
| 1 | Vehicle | 0 | 60 | Male | 6.7 | 0 | 0 |
| | control | | | Female | 6.0 | 0 | |
| | | | | Combined | 6.4 | 0 | |
| 2 | Low dose | 0.12 | 60 | Male | 6.5 | 0.121 | 0.031 |
| | | | | Female | 6.1 | 0.123 | |
| | | | | Combined | 6.3 | 0.122 | |
| 3 | Mid dose | 0.48 | 60 | Male | 6.9 | 0.505 | 0.128 |
| | | | | Female | 6.0 | 0.519 | |
| | | | | Combined | 6.5 | 0.512 | |
| 4 | High dose | 0.12 | 60 | Male | 6.9 | 1.325 | 0.335 |
| | | | | Female | 6.1 | 1.357 | |
| | | | | Combined | 6.5 | 1.341 | |

^{a}Calculated using the mean bodyweights obtained from Days −1 to 28.
^{b}Calculated using mean achieved aerosol concentrations from Days 1 to 28.
^{c}Calculated based on FDA assumed deposition fraction of 25% of mean estimated achieved doses within the respiratory tract.

The overall estimated achieved doses for all riboflavin 5′-phosphate treated groups were acceptable and within 11.7% of the projected dose levels. For toxicokinetic evaluation, the mean body weights and the achieved riboflavin 5′-phosphate aerosol concentrations from each respective toxicokinetic day were used to calculate the achieved dose levels. Table 18 summarizes achieved dose levels of riboflavin 5′-phosphate on day 1

TABLE 18

| | | Projected | | | | Mean | Mean |
|---|---|---|---|---|---|---|---|
| | | dose level | Duration of | | Mean body | estimated | achieved |
| Group | Group | of Riboflavin | exposure | | weight | achieved dose | respirable dose |
| Number | Designation | 5′-phosphate | (min) | Sex | (kg)^{a} | (mg/kg/day)^{b} | (mg/kg/day)^{c} |
| 1 | Vehicle | 0 | 60 | Male | 6.7 | 0 | 0 |
| | control | | | Female | 5.7 | 0 | |
| | | | | Combined | 6.2 | 0 | |
| 2 | Low dose | 0.12 | 60 | Male | 6.3 | 0.133 | 0.033 |
| | | | | Female | 5.9 | 0.134 | |
| | | | | Combined | 6.1 | 0.134 | |
| 3 | Mid dose | 0.48 | 60 | Male | 6.8 | 0.554 | 0.141 |
| | | | | Female | 5.8 | 0.571 | |
| | | | | Combined | 6.3 | 0.563 | |
| 4 | High dose | 1.20 | 60 | Male | 6.5 | 1.382 | 0.349 |
| | | | | Female | 5.9 | 1.408 | |
| | | | | Combined | 6.2 | 1.395 | |

^{a}Calculated using the mean body weights obtained from Day −1.
^{b}Calculated using mean achieved aerosol concentrations from Day 1.
^{c}Calculated based on FDA assumed deposition fraction of 25% of mean estimated achieved doses within the respiratory tract.

Table 19 summarizes achieved dose levels of riboflavin 5′-phosphate on day 28

TABLE 19

| | | Projected | | | | Mean | Mean |
|---|---|---|---|---|---|---|---|
| | | dose level | Duration of | | Mean body | estimated | achieved |
| Group | Group | of Riboflavin | exposure | | weight | achieved dose | respirable dose |
| Number | Designation | 5′-phosphate | (min) | Sex | (kg)^{a} | (mg/kg/day)^{b} | (mg/kg/day)^{c} |
| 1 | Vehicle | 0 | 60 | Male | 6.8 | 0 | 0 |
| | control | | | Female | 6.1 | 0 | |
| | | | | Combined | 6.5 | 0 | |
| 2 | Low dose | 0.12 | 60 | Male | 6.7 | 0.133 | 0.034 |
| | | | | Female | 6.3 | 0.135 | |
| | | | | Combined | 6.5 | 0.134 | |
| 3 | Mid dose | 0.48 | 60 | Male | 7.1 | 0.531 | 0.135 |
| | | | | Female | 6.1 | 0.547 | |
| | | | | Combined | 6.6 | 0.539 | |
| 4 | High dose | 1.20 | 60 | Male | 7.2 | 1.339 | 0.340 |
| | | | | Female | 6.2 | 1.378 | |
| | | | | Combined | 6.7 | 1.358 | |

^{a}Calculated using the mean body weights obtained from Day 28.
^{b}Calculated using mean achieved aerosol concentrations from Day 28.
^{c}Calculated based on FDA assumed deposition fraction of 25% of mean estimated achieved doses within the respiratory tract.

### Particle Size Analysis

Particle size distribution measurements are summarized in Table 20.

TABLE 20

| Group | Group | Mean Riboflavin 5′-phosphate Particle Size Data | | |
|---|---|---|---|---|
| Number | Designation | MMAD | σg | % of particles < 3.1 μm |
| 2 | Low Dose | 1.1 | 2.02 | 91.1 |
| 3 | Mid Dose | 1.1 | 2.01 | 93.3 |
| 4 | High Dose | 1.2 | 2.11 | 87.2 |

* For Group 1 (Vehicle Control), a MMAD of 1.4 μm with a σg of 2.08 μm was obtained by gravimetric determination
MMAD = Mass median aerodynamic diameter (μm)
σg = Geometric standard deviation.

Particle size distribution measurements confirmed that the aerosolized formulations of riboflavin 5′-phosphate were respirable for the dog, and the deposition within the respiratory tract was considered to be 100%.

### Relative Humidity, Temperature and Oxygen Concentration

Exposure atmosphere oxygen concentrations, temperature and relative humidity for the duration of the study are summarized in Table 21.

TABLE 21

| | Group | | | | | |
|---|---|---|---|---|---|---|
| Group | Desig- | Relative humidity (%) | | Temperature (%) | | Oxygen |
| Number | nation | Min | Max | Min | Max | (%) |
| 1 | Vehicle | 40.1 | 74.3 | 21.1 | 22.7 | 20.9 |
| | control | | | | | |
| 2 | Low dose | 49.2 | 79.5 | 21.6 | 23.4 | 20.9 |
| 3 | Mid dose | 40.7 | 74.2 | 21.5 | 22.5 | 20.9 |
| 4 | High dose | 49.8 | 81.5 | 21.6 | 23.4 | 20.9 |

Exposure atmosphere oxygen concentrations, temperature and relative humidity ranges were considered acceptable.

### In-Life Observations

### Mortality

There were no premature deaths during the course of this study.

### Clinical Signs

There were no clinical signs related to treatment with Riboflavin 5′-phosphate observed during the treatment period. During exposure, vocalization, increased respiration, increased activity and salivation were noted across treated and control groups. These clinical signs were transient in duration and are commonly observed in dogs as a consequence of the stress associated with restraint for the inhalation exposures. Hence, these clinical signs were not considered test article-related. Clinical observations were considered to be incidental and unrelated to treatment.

### Body Weight

There were no changes in body weights that were considered related to treatment with Riboflavin 5′-phosphate.

### Food Consumption

There were no changes noted in food consumption that were considered to be treatment-related. Statistically significant differences (p≦0.05) from the vehicle control group were noted in a number of days. However, the differences from the vehicle control group were inconsistent between sexes and showed no relationship to dose level and were considered incidental and unrelated to treatment.

### Ophthalmology

There were no ocular changes noted that were considered to be treatment-related. There were unilateral or bilateral ocular findings such as persistent papillary membrane, tapetal pigmentation variation, nuclear punctuate opacities and prominent posterior suture lines noted at the end of treatment. However, these findings were observed at the pretreatment ophthalmic evaluation, were incidental and considered normal for the dog population and were unrelated to treatment with riboflavin 5′-phosphate.

### Electrocardiography

Electrocardiogram wave forms were unaffected by treatment with riboflavin 5′-phosphate. The data obtained for this parameter does not support a treatment related effect of either the control or the test article (riboflavin 5′-phosphate). There were no changes in blood pressure that were considered related to treatment with riboflavin 5′-phosphate.

### Respiratory Parameters Measurements

There were no changes in respiratory parameters evaluated (respiratory rate, tidal volume and minute volume) that were considered affected by treatment with riboflavin 5′-phosphate. Statistically significant differences (p≦0.05) from the vehicle control group were noted in the respiratory rate in 2 treated groups. However, statistically significant the changes were not considered of any biological significance and were within the expected range.

### Toxicokinetics

Riboflavin 5′-phosphate concentrations were detected in only a single treated animal (Group 2) following single or repeated exposures to riboflavin 5′-phosphate. Since no riboflavin 5′-phosphate was detected in any other treated animals no kinetics were calculated.

### Day 1

There were no measurable concentrations of riboflavin 5′-phosphate and only 6 measurable concentrations of its metabolite (riboflavin) in the control group (Group 1) on Day 1, with concentrations only slightly above the quantifiable limit. The median Tₘₐₓ value of riboflavin was 0 hour (i.e. Cₘₐₓ was reached at the immediately post dosing time point) for all groups except Group 2 males which showed a median Tₘₐₓ value of 0.125 hours. Mean Cₘₐₓ values of riboflavin were 0.0588 μg/mL, 0.205 μg/mL and 0.358 μg/mL for males and 0.0566 μg/mL, 0.161 μg/mL and 0.391 μg/mL for females for low, mid and high dose groups, respectively. The corresponding AUC_{(0-T)} values were 0.0344 μg·h/mL, 0.295 μg·h/mL and 0.588 μg·h/mL for males and 0.151 μg·h/mL, 0.430 μg·h/mL and 0.625 μg·h/mL for females, respectively. Day 1 plasma exposures of riboflavin (Cₘₐₓ and AUC_{(0-T)}) increased as a function of the administered doses of riboflavin 5′-phosphate in both sexes and there was no observed apparent gender related difference of riboflavin plasma exposure (Cₘₐₓ and AUC_{(0-T)}) following a single administration of riboflavin 5′-phosphate.

### Day 28

There were 3 measurable concentrations of riboflavin 5′-phosphate and 8 measurable concentrations of its metabolite (riboflavin) in the control group (Group 1) on Day 28, with concentrations only slightly above the quantifiable limits. Cₘₐₓ values of riboflavin on Day 28 were 0.0623 μg/mL, 0.140 μg/mL and 0.382 μg/mL for males and 0.0498 μg/mL, 0.147 μg/mL and 0.289 μg/mL for females for low, mid and high dose groups, respectively. The corresponding AUC_{(0-T)} values were 0.0582 μg·h/mL, 0.243 μg·h/mL and 0.554 μg·h/mL for males and 0.0846 μg·h/mL, 0.773 μg·h/mL and 0.509 μg·h/mL for females, respectively. Day 28 plasma exposures of riboflavin (Cₘₐₓ and AUC_{(0-T)}) increased as a function of the administered doses of riboflavin 5′-phosphate in both sexes. Overall, the Cₘₐₓ and AUC_{(0-T)} values of riboflavin on Day 28 were comparable to those of Day 1 in both sexes. Cₘₐₓ Day 28 to Day 1 Cₘₐₓ ratios of riboflavin were 1.06, 0.683 and 1.07 in males and 0.880, 0.913 and 0.739 in females for the low, mid and high dose groups. The corresponding AUC_{(0-T)} ratios of riboflavin were 1.69, 0.824 and 0.942 in males and 0.560, 1.80 and 0.814 in females, respectively.

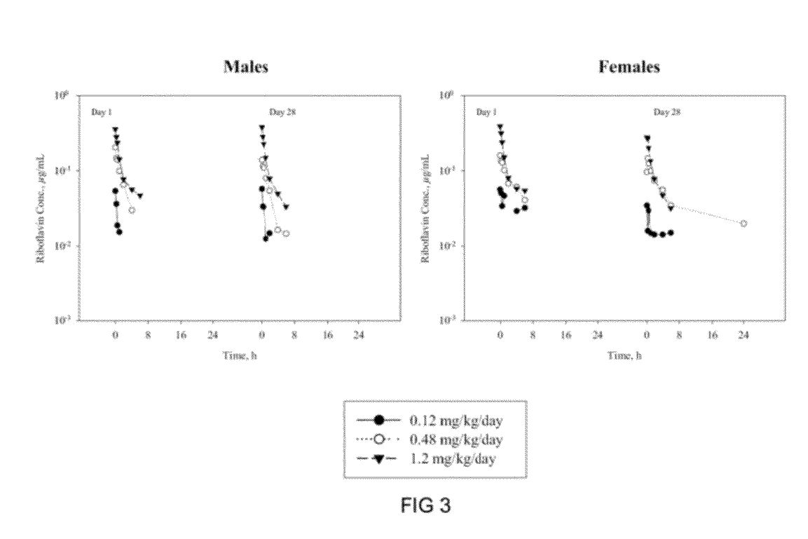

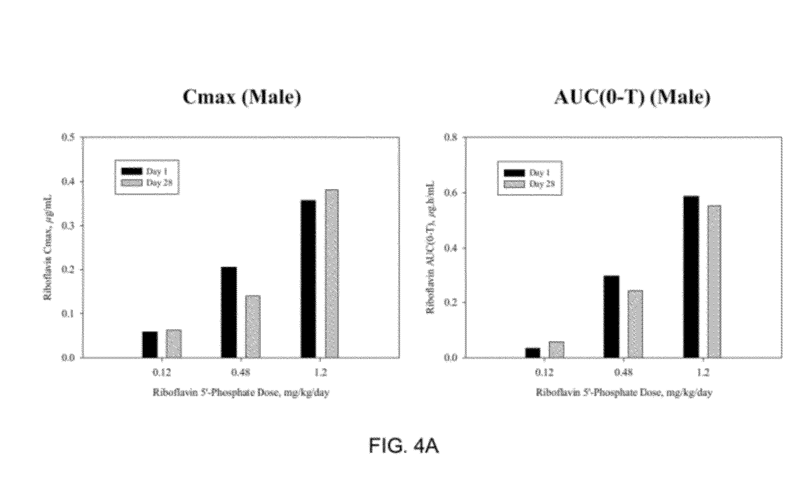

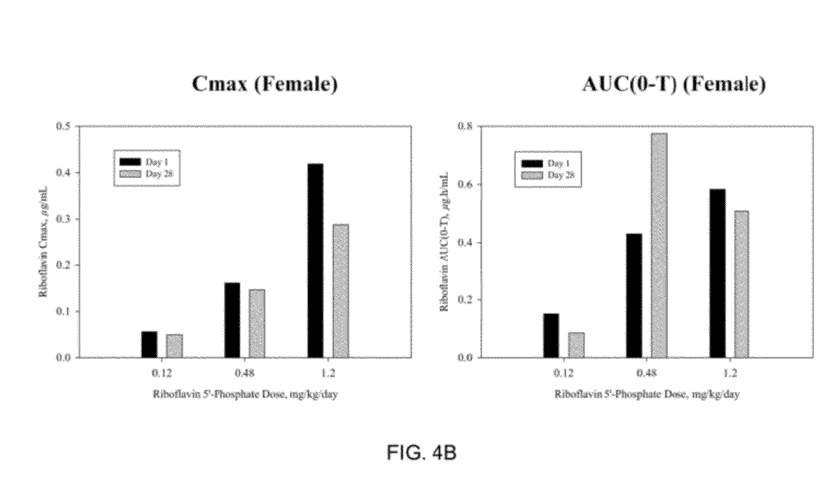

FIG. 3 shows graphs of mean riboflavin plasma concentration-time profiles in male and female dogs following aerosolized doses of riboflavin 5′-phosphate for 28 Days. FIG. 4A and FIG. 4B show graphs for mean riboflavin Cₘₐₓ and AUC_{(0-T)} in dogs following aerosolized doses of riboflavin 5′-phosphate for 28 days. The following table summarizes toxicokinetic parameters of riboflavin in dogs following aerosolized doses of riboflavin 5′-phosphate for 28 Days.

TABLE 22

| | Riboflavin | Male | | | Female | | |
|---|---|---|---|---|---|---|---|
| Day | 5′-phosphate | 0.12 | 0.48 | 1.2 | 0.12 | 0.48 | 1.2 |
| 1 | Cₘₐₓ (μg/ml) | 0.0588 | 0.205 | 0.358 | 0.0566 | 0.161 | 0.391 |
| | Tmax (h)^{a} | 0.125 | 0.00 | 0.00 | 0.00 | 0.00 | 0.00 |
| | AUC(0-T) | 0.0344 | 0.295 | 0.588 | 0.151 | 0.430 | 0.625 |
| | (μg · h/ml) | | | | | | |
| | Cmax ratio | 1.04 | 1.27 | 0.916 | — | — | — |
| | (M/F)^{b} | | | | | | |
| | AUC ratio | 0.228 | 0.686 | 0.941 | — | — | — |
| | (M/F)^{c} | | | | | | |
| 28 | Cₘₐₓ (μg/ml) | 0.0623 | 0.140 | 0.382 | 0.0498 | 0.147 | 0.289 |
| | Tmax (h)^{a} | 0.00 | 0.00 | 0.00 | 0.00 | 0.250 | 0.00 |
| | AUC(0-T) | 0.0582 | 0.243 | 0.554 | 0.0846 | 0.773 | 0.509 |
| | (μg · h/ml) | | | | | | |
| | Cmax ratio | 1.06 | 0.683 | 1.07 | 0.880 | 0.913 | 0.739 |
| | (M/F)^{d} | | | | | | |
| | AUC ratio | 1.69 | 0.824 | 0.942 | 0.560 | 1.80 | 0.814 |
| | (M/F)^{e} | | | | | | |

^{a}Expressed as median and range
^{b}Cₘₐₓmale/Cₘₐₓfemale
^{c}AUC_{(0-T)}male/AUC_{(0-T)}female
^{d}CₘₐₓDay 28/CₘₐₓDay 1
^{e}AUC_{(0-T)}Day 28/AUC_{(0-T)}Day 1

### Clinical Pathology

### Hematology

There were no changes noted in hematology parameters that were considered to be treatment-related during the treatment period. Parameters measured were the same as those listed in Example 1. Statistically significant differences (p≦0.05) from the vehicle control group were noted in a number of parameters. However, the differences from the vehicle control group were inconsistent between sexes and showed no relationship to dose level and were considered incidental and unrelated to treatment.

### Coagulation

Coagulation times were unaffected by treatment. Parameters measured included prothrombin time (PTT) using a coagulometric method, and activated partial thromboplastin time (APTT) using a coagulometric method. The following table summarizes coagulation parameters before treatment and at Day 29.

### Clinical Chemistry

There were no changes noted in clinical chemistry parameters that were considered to be treatment-related during the treatment period. Statistically significant differences (p≦0.05) from the vehicle control group were noted in a number of parameters. However, the differences from the vehicle control group were inconsistent between sexes and showed no relationship to dose level and were considered incidental and unrelated to treatment.

### Urinalysis

There were no changes in any of the urinalysis parameters evaluated that were considered affected by treatment. Statistically significant differences (p≦0.05) from the vehicle control group were noted in some parameters. However, the differences from the vehicle control group were inconsistent between sexes and showed no relationship to dose level and were considered incidental and unrelated to treatment.

### Organ Weights

A slight increase in spleen weight was observed in Group 4 males (both absolute and relative to body weight) and Group 3 males (relative to body weight) at termination of the treatment period. However, the weights were within expected limits and the statistical significance was considered to be due to the lighter control weights recorded in all 3 control males and no such weight difference was recorded in corresponding females. Differences from control weights recorded in other tissues, occasionally attaining statistical significance, were also considered unrelated to treatment and of no biological significance.

### Macroscopic Findings

There were no macroscopic findings that were considered to be test article related. Sporadic findings in the lungs were noted infrequently in most treatment groups, including controls, and were not considered test article related. Small prostate and thymus were age related incidental findings and not test article related. Other changes were considered procedure-related, agonal, or incidental and not test article-related.

### Microscopic Findings

There was no evidence of test article-related histopathological finding at termination of the treatment period. Chronic active inflammation and bronchioalveolar inflammation of the lungs, frequently associated with foreign body granulomas, were seen occasionally in most treatment groups, including controls, and were not considered test article related. Other changes were not considered toxicologically significant as they were agonal, not dose-related, of low incidence or severity, occurred in control and treated animals, or are incidental in this age and strain of beagle dog.

### Discussion

The objective of the study was to determine the toxicity and the toxicokinetic profile of the test article, riboflavin 5′-phosphate, following oronasal inhalation administration to the beagle dog for 28 days. Inhalation exposure to riboflavin 5′-phosphate was well tolerated in beagle dogs and there were no premature deaths during the course of the study. No adverse clinical observations, ocular effects, electrocardiogram wave forms, indirect blood pressure readings, respiratory measurements or systemic effects detected by assessment of clinical pathology were noted in any of the groups. There were no macroscopic or microscopic findings that were considered to be related to the test article, riboflavin 5′-phosphate. Due to the lack of measurable plasma concentrations of riboflavin 5′-phosphate in treated animals no kinetics were calculated. Concentrations of the metabolite (riboflavin) were measurable in the plasma of both sexes and exposures (Cₘₐₓ and AUC_{(0-T)}) appeared to be dose proportional. Overall, there was no gender difference in riboflavin exposure and there was no accumulation following repeated dosing of riboflavin 5′-phosphate for 28 days. In conclusion, the No Observed Effect Level (NOEL) in Beagle dogs after inhalation (oronasal) administration of riboflavin 5′-phosphate up to 60 minutes/day for 28 consecutive days, is considered to be at the high-dose group tested at 1.341 mg/kg/day.

### Example 3

### Phase 2 Clinical Study: Administration of Levofloxacin Formulated with MgCl₂ to COPD Patients or Placebo Comprising Riboflavin 5′-Phosphate

A clinical study to evaluate the safety, tolerability and efficacy of levofloxacin formulated with MgCl₂ was carried out on COPD patients. The study is a Phase 2, multi-center, randomized, double-blind, placebo-controlled study. Patients are administered 240 mg BID levofloxacin formulated with MgCl₂ or placebo which included riboflavin 5′-phosphate. The formulations for the study drug and placebo are shown in Table 23.

TABLE 23

| | Levofloxacin formulated | |
|---|---|---|
| | with MgCl₂ | Placebo |
| Levofloxacin, mg/ml (mM) | 100 (272) | 0 |
| Magnesium, mg/ml (mM) | 4.9 (200) | 0 |
| Chloride, mg/ml (mM) | 14.2 (400) | 0 |
| pH | 6-8 | 4.5-7.5 |
| Osmolality, mOsm/kg | 300-500 | 270-300 |
| Saline | N/A | 0.9% |
| Riboflavin 5′-phosphate, μg/ml | 0 | 4 |

Study drug and placebo were administered using a modified PARI eFlow® nebulizer. The study included a series of at least six, but no more than 12, treatment cycles. Each treatment cycle was 28 days. In each treatment cycle, patients were administered either 240 mg BID levofloxacin with MgCl₂ or placebo for 5 consecutive days.

### Patient Population

Approximately 300 patients were studied. Criteria for including patients in this study include those that have: (1) a history of COPD with mucopurulent sputum (yellow, green or brown/tan) production on most days, even when exacerbation-free; (2) a measured FEV₁<70% of predicted FEV₁ (post-bronchodilator administration) and FEV₁/FVC<0.7 (post-bronchodilator) at screening based on predicted values using age, height and sex using Hankinson and N. Hanes criteria; (3) had at least two documented acute exacerbation episodes during the preceding 12 months prior to Day 1 of Cycle 1, acute exacerbation episodes include episodes that require antibiotic agents, systemic corticosteroids, hospitalization or a combination of these treatments; (4) had no acute exacerbation episode that required treatment within 30 days prior to Day 1 of Cycle 1; (5) a stable treatment history for 30 days prior to Day 1 of Cycle 1, if the patient is receiving chronic therapy with inhaled long acting bronchodilators and/or inhaled or systemic steroids; and (6) a lifetime smoking history of at least 10 pack-years. Criteria for excluding patients from this study included those that have any respiratory tract disorder other than COPD that was considered to be clinically relevant, for example, a history of a primary diagnosis of asthma, bronchial carcinoma, pulmonary tuberculosis, cystic fibrosis or diffuse bronchiectasis.

The patient population included an efficacy evaluable (EE) population, a modified intent to treat (MITT) population, and a pharmacokinetic (PK) population. The EE population included all patients enrolled in the study who complete 80% of their treatment cycles without major protocol violations. The MITT population included all patients enrolled in the study that receive at least one dose of study drug. The PK population included all patients that receive a least one dose of Study Drug and have at least one PK sample collected.

### Study Endpoints

Efficacy was evaluated using: (1) the durations and severity of any exacerbation events; (2) sputum microbiology; (3) pulmonary function tests; (4) quality of life/symptoms and signs; and (5) the BODE index.

### Exacerbations

Acute exacerbations of COPD were a primary endpoint of the study. An acute exacerbation includes a symptomatic respiratory deterioration requiring treatment with antibiotic agents, systemic corticosteroids, hospitalization, or a combination of these treatments. In addition, exacerbations may be characterized by increased sputum production, more purulent sputum, change in sputum color, increased coughing, increased wheezing, chest tightness, reduced exercise tolerance, increased fatigue, fluid retention, acute confusion, worsened dyspnea.

The severity of an acute exacerbation was measured using criteria that include, for example, the medication required, dose of medication required, date of onset of an acute exacerbation, and duration of the acute exacerbation. The primary efficacy analysis includes a comparison of exacerbation rates between the levofloxacin treatment group and the placebo treatment group, and/or a comparison of the severity of any exacerbation between the levofloxacin treatment group and the placebo treatment group.

### Microbiological Evaluations

Bacteria were identified and quantified in patients' sputum. Sputum from COPD patients administered levofloxacin formulated with MgCl₂ had have a lower density of bacteria, including S. pneumoniae, B-hemolytic streptococci, S. aureus, H. influenzae, M. catarrhalis, P. aeruginosa, and other enterobacteriaceae, compared to sputum from COPD patients administered placebo. Reduced densities of bacteria were observed in patients administered levofloxacin.

### Pulmonary Function Evaluations

All patients underwent pulmonary function testing to determine their forced vital capacity (FVC) and forced expiratory volume in one second (FEV₁). FEV₁/FVC ratio, FVC percent predicted, and FEV₁ percent predicated. Pulmonary function tests were performed according to American Thoracic Society/European Respiratory Society (ATS/ERS) Spirometry Standards (2005), incorporated by reference in its entirety.

### Quality of Life/Signs and Symptoms

Patients completed a St. George's Respiratory Questionnaire (SGRQ), sign and symptoms questionnaire (Meguro et al., (2007) “Development and validation of an improved, COPD-specific version of the St George's Respiratory Questionnaire” Chest 132:456-63, incorporated by reference in its entirety). SGRQ is a disease-specific instrument designed to measure impact on overall health, daily life, and perceived well-being and has been developed for use by patients with fixed and reversible airway obstruction. Scores for these components and the summary score were based on a 100-point scale.

### BODE Index

The BODE index is a multidimensional grading system that assesses the respiratory, perceptive, and systemic aspects of COPD that would better categorize the illness. The index relates to a body-mass index (B), the degree of airflow obstruction (O), functional dyspnea (D), and exercise capacity (E) (B-O-D-E) (Celli B R, et al., The body-mass index, airflow obstruction, dyspnea, and exercise capacity index in chronic obstructive pulmonary disease. N Engl J. Med. 2004 Mar. 4; 350 (10):1005-12, incorporated by reference in its entirety). Accordingly, the severity of COPD and the risk of death in patients with COPD may be graded using variables that include FEV₁, the presence of hypoxemia or hypercapnea, measurements from a short distance walked in a fixed time, the degree of functional breathlessness, and body-mass index.

Patients were assessed measuring factors of the BODE index including the six minute walk test (6MWT). The 6MWT measures the distance that a patient can walk at his/her pace on a measured surface in 6 minutes.

While preferred embodiments of the present invention have been shown and described herein, it will be obvious to those skilled in the art that such embodiments are provided by way of example only. Numerous variations, changes, and substitutions will occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

All publications, patents, and patent applications mentioned in this specification are herein incorporated by reference to the same extent as if each individual publication, patent, or patent application was specifically and individually indicated to be incorporated by reference.

What is claimed:

1. A method for evaluating a test compound comprising:
administering to a first population of individuals a test compound via inhalation of an aerosol; administering to a second population of individuals a placebo comprising riboflavin 5′-phosphate via inhalation of an aerosol; and
comparing a biological marker in at least one individual administered the test compound to a biological marker in at least one individual administered the placebo.

2. The method of claim 1, wherein the administering the test compound comprises delivering an aerosolized solution of the test compound.

3. The method of claim 1, wherein the administering is intrapulmonary or intranasal.

4. The method of claim 1, wherein the test compound or placebo are delivered with a pulmonary delivery device.

5. The method of claim 1, wherein the biological marker is selected from the group consisting of a marker associated with a therapeutic effect, a marker associated with an adverse effect, a marker associated with a toxic effect, a marker associated with a pharmacodynamic parameter.

6. The method of claim 1, wherein the test compound comprises at least one member of the group consisting of antibiotics, antiallergics, anticancer agents, antifungals, antineoplastic agents, analgesics, bronchodilators, antihistamines, antiviral agents, antitussives, anginal preparations, anti-inflammatories, immunomodulators, 5-lipoxygenase inhibitors, leukotriene antagonists, phospholipase A2 inhibitors, phosphodiesterase IV inhibitors, peptides, proteins, steroids, and vaccine preparations.

7. The method of claim 1, wherein the placebo comprises a solution of riboflavin 5′-phosphate.

8. The method of claim 7, wherein the solution of riboflavin 5′-phosphate comprises a concentration greater than about 0.1 mg/ml.

9. The method of claim 7, wherein the solution of riboflavin 5′-phosphate comprises a concentration greater than about 0.06 mg/L.

10. The method of claim 1, wherein the riboflavin 5′-phosphate aerosol comprises a dose greater than about 0.01 mg/kg/day.

11. The method of claim 1, wherein the riboflavin 5′-phosphate aerosol comprises a dose greater than about 2.0 mg/kg/day.

12. The method of claim 1, wherein the aerosolized riboflavin 5′-phosphate comprises a mass median aerodynamic diameter from about 0.5 μm to about 4.5 μm with a geometric standard deviation less than or equal to 3.0 μm.

13. The method of claim 1, wherein the aerosolized riboflavin 5′-phosphate comprises a mass median aerodynamic diameter from about 1.0 μm to about 3.5 μm with a geometric standard deviation less than or equal to 2.7 μm.

14. The method of claim 1, wherein the aerosolized riboflavin 5′-phosphate comprises a mass median aerodynamic diameter from about 1.1 μm to about 3.1 μm with a geometric standard deviation less than or equal to 2.4 μm.

15. The method of claim 1, wherein the individuals are animals.

16. A method for evaluating a test compound comprising: conducting a drug trial of a test compound and a placebo in a population of individuals, wherein the placebo comprises aerosolized riboflavin 5′-phosphate.

17. An aerosol comprising riboflavin 5′-phosphate.

18. The aerosol of claim 17 comprising a solution of riboflavin 5′-phosphate.

19. The aerosol of claim 18, wherein the solution of riboflavin 5′-phosphate comprises a concentration greater than about 0.1 mg/ml.

20. The aerosol of claim 18, wherein the solution of riboflavin 5′-phosphate comprises a concentration greater than about 0.06 mg/L.

21. The aerosol of claim 17, wherein the aerosol comprises a dose of riboflavin 5′-phosphate greater than about 0.01 mg/kg/day.

22. The aerosol of claim 17, wherein the aerosol comprises a dose of riboflavin 5′-phosphate greater than about 2.0 mg/kg/day.

23. The aerosol of claim 17, wherein the aerosolized riboflavin 5′-phosphate comprises a mass median aerodynamic diameter from about 0.5 μm to about 4.5 μm with a geometric standard deviation less than or equal to 3.0 μm.

24. The aerosol of claim 17, wherein the aerosolized riboflavin 5′-phosphate comprises a mass median aerodynamic diameter from about 1.0 μm to about 3.5 μm with a geometric standard deviation less than or equal to 2.7 μm.

25. The aerosol of claim 17, wherein the aerosolized riboflavin 5′-phosphate comprises a mass median aerodynamic diameter from about 1.1 μm to about 3.1 μm with a geometric standard deviation less than or equal to 2.4 μm.

26. The aerosol of claim 18, wherein the aerosol comprises a dose of riboflavin 5′-phosphate greater than about 0.01 mg/kg/day.

27. The aerosol of claim 18, wherein the aerosol comprises a dose of riboflavin 5′-phosphate greater than about 2.0 mg/kg/day.

28. The aerosol of claim 18, wherein the aerosolized riboflavin 5′-phosphate comprises a mass median aerodynamic diameter from about 0.5 μm to about 4.5 μm with a geometric standard deviation less than or equal to 3.0 μm.

29. The aerosol of claim 18, wherein the aerosolized riboflavin 5′-phosphate comprises a mass median aerodynamic diameter from about 1.0 μm to about 3.5 μm with a geometric standard deviation less than or equal to 2.7 μm.

30. The aerosol of claim 18, wherein the aerosolized riboflavin 5′-phosphate comprises a mass median aerodynamic diameter from about 1.1 μm to about 3.1 μm with a geometric standard deviation less than or equal to 2.4 μm.
